(19) United States
(12) Patent Application Publication
(10) Pub. No.: US 2010/0104548 A1
(43) Pub. Date: Apr. 29, 2010

(54) MODULATION OF AMINO ACID METABOLISM IN THE HYPOTHALAMUS

(75) Inventors: Luciano Rossetti, Bedford Hills, NY (US); Tony Lam, Richmond Hill (CA); Roger Gutierrez-Juarez, Bronx, NY (US)

Correspondence Address: AMSTER, ROTHSTEIN & EBENSTEIN LLP, 90 PARK AVENUE, NEW YORK, NY, 10016, US

(73) Assignees: ALBERT EINSTEIN COLLEGE OF MEDICINE OF YESHIVA UNIVERSITY, Bronx, NY (US); SIGNUM PHARMACEUTICALS, INC., Raleigh, NC (US)

(21) Appl. No.: 11/921,847

(22) PCT Filed: Jun. 21, 2006
(86) PCT No.: PCT/US2006/024430

§ 371 Date: Jan. 4, 2010

Related U.S. Application Data
(60) Provisional application No. 60/694,111, filed on Jun. 24, 2005.

(51) Int. Cl. A61K 31/401; A61K 31/195; A61K 38/44; A61K 38/45; A61P 3/00

(52) U.S. Cl. 424/94.4; 514/423; 514/561; 424/94.5

(57) ABSTRACT

Methods for reducing peripheral blood glucose levels, food intake, glucose production, gluconeogenesis, triglyceride levels, and low density lipoprotein (VLDL) levels in mammals are provided. Also provided are methods of increasing glucose production and food intake in mammals. Further provided are methods of treating a disorder selected from the group consisting of obesity, type 2 diabetes, type 1 diabetes, hyperglycemia, insulin resistance, glucose intolerance, leptin resistance, metabolic syndrome, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, and any combination of the foregoing. The methods involve manipulations of amino acid presence or metabolism in the hypothalamus of the mammal.

### RELATED APPLICATION INFORMATION

This application claims the benefit of U.S. Provisional Application Ser. No. 60/694,111, filed Jun. 24, 2005, the disclosure of which is incorporated herein by reference in its entirety.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of DK 48321, DK 45024, DK 066058, and DK 47208 awarded by the National Institutes of Health.

### BACKGROUND OF THE INVENTION

(1) Field of the Invention

The present invention generally relates to methods for regulating food intake, glucose production, triglyceride levels and/or lipoprotein levels. More specifically, the invention relates to regulation of food intake, glucose production, triglyceride levels and/or lipoprotein levels by manipulating amino acid levels and metabolism in the hypothalamus.

(2) Description of the Related Art

References Cited
Ahima, R S, Prabakaran, D, Mantzoros, C, Qu, D, Lowell, B, Maratos-Flier, E, Flier, J S: Role of leptin in the neuroendocrine response to fasting. Nature 382:250-252, 1996
Air, E L, Strowski, M Z, Benoit, S C, Conarello, S L, Salituro, G M, Guan, X M, Liu, K, Woods, S C, Zhang, B B: Small molecule insulin mimetics reduce food intake and body weight and prevent development of obesity. Nat. Med. 8:179-183, 2002
Brooks, G A, Dubouchaud, H, Brown, M, Sicurello, J P, Butz, C E: Role of mitochondrial lactate dehydrogenase and lactate oxidation in the intracellular lactate shuttle. Proc.Natl.Acad.Sci. U.S.A 96:1129-1134, 1999
Bruning, J C, Michael, M D, Winnay, J N, Hayashi, T, Horsch, D, Accili, D, Goodyear, L J, Kahn, C R: A muscle-specific insulin receptor knockout exhibits features of the metabolic syndrome of NIDDM without altering glucose tolerance. Mol.Cell 2:559-569, 1998
Friedman, J M: A war on obesity, not the obese. Science 299:856-858, 2003
Harris, R A, Paxton, R, DePaoli-Roach, A A: Inhibition of branched chain alpha-ketoacid dehydrogenase kinase activity by alpha-chloroisocaproate. J.Biol.Chem. 257:13915-13918, 1982
Hill, J O, Peters, J C: Environmental contributions to the obesity epidemic. Science 280:1371-1374, 1998
Kopelman, P G, Hitman, G A: Diabetes. Exploding type II. Lancet 352 Suppl 4:SIV5, 1998
Lam, T K, Pocai, A, Gutierrez-Juarez, R, Obici, S, Bryan, J, Aguilar-Bryan, L, Schwartz, G J, Rossetti, L: Hypothalamic sensing of circulating fatty acids is required for glucose homeostasis. Nat. Med. 11:320-327, 2005
Obici, S, Zhang, B B, Karkanias, G, Rossetti, L: Hypothalamic insulin signaling is required for inhibition of glucose production. Nat. Med. 2002
Obici, S, Feng, Z, Arduini, A, Conti, R, Rossetti, L: Inhibition of hypothalamic carnitine palmitoyltransferase-1 decreases food intake and glucose production. Nat. Med. 9:756-761, 2003
Pellerin, L, Magistretti, P J: Neuroscience. Let there be (NADH) light. Science 305:50-52, 2004
Schwartz, M W, Woods, S C, Porte, D, Jr., Seeley, R J, Baskin, D G: Central nervous system control of food intake. Nature 404:661-671, 2000
Wang, J, Liu, R, Hawkins, M, Barzilai, N, Rossetti, L: A nutrient-sensing pathway regulates leptin gene expression in muscle and fat. Nature 393:684-688, 1998
Woods, S C, Lotter, E C, McKay, L D, Porte, D, Jr.: Chronic intracerebroventricular infusion of insulin reduces food intake and body weight of baboons. Nature 282:503-505, 1979
PCT Patent Application PCT/US2004/004344, filed Feb. 12, 2004
PCT Patent Application No. PCT/US04/16562, filed May 27, 2004
U.S. patent application Ser. No. 11/353,594, filed Feb. 13, 2006
PCT Patent Application PCT/US2006/005045, filed Feb. 13, 2006
PCT Patent Application No. PCT/US06/16967, filed May 2, 2006

Obesity and type 2 diabetes mellitus are due to the impact of environmental factors in individuals with an underlying genetic susceptibility (Hill and Peters, 1998; Kopelman and Hitman, 1998). Hypothalamic centers can monitor the availability of circulating nutrients via nutrient-induced peripheral signals such as leptin and insulin (Ahima et al., 1996; Air et al., 2002; Bruning et al., 1998; Friedman, 2003; Schwartz et al., 2000; Wang et al., 1998; Woods et al., 1979) as well as via direct metabolic signaling (Obici et al., 2002; Obici et al., 2003; Lam et al., 2005; Pellerin and Magistretti, 2004). In this regard, the metabolism of both lipids and carbohydrates in selective hypothalamic neurons has recently been shown to function as a primary biochemical sensor for nutrient availability, which in turn exerts a negative feedback on food intake (Obici et al., 2002; Obici et al., 2003) and endogenous glucose production (Obici et al., 2003). See also PCT Patent Application PCT/US2004/004344, filed Feb. 12, 2004, U.S. patent application Ser. No. 11/353,594, filed Feb. 13, 2006, PCT Patent Application PCT/US2006/005045, filed Feb. 13, 2006, and PCT Patent Application No. PCT/US06/16967, filed May 2, 2006, all incorporated by reference in their entirety.

It would be desirable to further characterize hypothalamic signaling mechanisms affecting glucose production, food intake, triglyceride levels and/or lipoprotein levels. The present invention addresses that need.

### SUMMARY OF THE INVENTION

Accordingly, the inventors have discovered that increasing amino acid levels in the hypothalamus, or increasing amino acid metabolism toward acetyl-CoA in the hypothalamus causes a reduction in glucose production, food intake, very low density lipoprotein (VLDL) levels, and triglyceride levels in the mammal.

Thus, in some embodiments, the present invention is directed to methods of reducing peripheral glucose levels (e.g., in blood, plasma or serum) in a mammal. The methods comprise administering a compound to the mammal, where administering the compound to the mammal reduces peripheral blood glucose levels in the mammal and

(a) directly causes an increase in an amino acid in the hypothalamus of the mammal, or

(b) causes an increase in metabolism of the amino acid or an α-keto acid corresponding to the amino acid toward acetyl-CoA in the hypothalamus of the mammal. In these embodiments, the compound is not pyruvate.

In other embodiments, the invention is directed to additional methods of reducing peripheral glucose levels (e.g., in blood, plasma, or serum) in a mammal. The methods comprise administering a compound to the mammal in an amount effective to reduce blood glucose levels in the mammal. In these embodiments, the compound is an amino acid, an immediate precursor to an amino acid, an amino acid analog, an amino acid-increasing molecule, a nucleic acid encoding an amino acid-increasing molecule, a molecule that metabolizes an amino acid toward acetyl-CoA, a nucleic acid encoding a molecule that metabolizes an amino acid toward acetyl-CoA, or an inhibitor of an enzyme that decreases the activity of a molecule that metabolizes the amino acid toward acetyl-CoA.

Additionally, the invention is directed to methods of reducing food intake in a mammal. The methods comprise administering a compound to the mammal, where administering the compound to the mammal reduces food intake in the mammal and

(a) directly causes an increase in an amino acid in the hypothalamus of the mammal, or

(b) causes an increase in metabolism of the amino acid or an α-keto acid corresponding to the amino acid toward acetyl-CoA in the hypothalamus of the mammal. In these embodiments, the compound is not pyruvate.

The present invention is further directed to additional methods of reducing food intake in a mammal. The methods comprise administering a compound to the mammal in an amount effective to reduce food intake in the mammal, where the compound is an amino acid, an immediate precursor to an amino acid, an amino acid analog, an amino acid-increasing molecule, a nucleic acid encoding an amino acid-increasing molecule, a molecule that metabolizes an amino acid toward acetyl-CoA, a nucleic acid encoding a molecule that metabolizes an amino acid toward acetyl-CoA, or an inhibitor of an enzyme that decreases the activity of a molecule that metabolizes the amino acid toward acetyl-CoA.

In further embodiments, the invention is directed to methods of reducing glucose production in a mammal. The methods comprise administering a compound to the mammal, where administering the compound to the mammal reduces glucose production in the mammal and

(a) directly causes an increase in an amino acid in the hypothalamus of the mammal, or

(b) causes an increase in metabolism of the amino acid or an α-keto acid corresponding to the amino acid toward acetyl-CoA in the hypothalamus of the mammal. In these embodiments, the compound is not pyruvate.

Additionally, the invention is directed to additional methods of reducing glucose production in a mammal. The methods comprise administering a compound to the mammal in an amount effective to reduce glucose production in the mammal, where the compound is an amino acid, an amino acid analog, an immediate precursor to an amino acid, an amino acid-increasing molecule, a nucleic acid encoding an amino acid-increasing molecule, a molecule that metabolizes an amino acid toward acetyl-CoA, a nucleic acid encoding a molecule that metabolizes an amino acid toward acetyl-CoA, or an inhibitor of an enzyme that decreases the activity of a molecule that metabolizes the amino acid toward acetyl-CoA.

In additional embodiments, the invention is directed to methods of inhibiting gluconeogenesis in the liver of a mammal. The methods comprise administering a compound to the mammal, where administering the compound to the mammal inhibits gluconeogenesis in the mammal and

(a) directly causes an increase in an amino acid in the hypothalamus of the mammal, or

(b) causes an increase in metabolism of the amino acid or an α-keto acid corresponding to the amino acid toward acetyl-CoA in the hypothalamus of the mammal. In these embodiments, the compound is not pyruvate.

The invention further provides a method of inhibiting gluconeogenesis in the liver of a mammal, the method comprising administering a compound to the mammal in an amount effective to inhibit gluconeogenesis in the liver of the mammal, wherein the compound is an amino acid, an amino acid analog, an immediate precursor to an amino acid, an amino acid-increasing molecule, a nucleic acid encoding an amino acid-increasing molecule, a molecule that metabolizes an amino acid toward acetyl-CoA, a nucleic acid encoding a molecule that metabolizes an amino acid toward acetyl-CoA, or an inhibitor of an enzyme that decreases the activity of a molecule that metabolizes the amino acid toward acetyl-CoA.

In other embodiments, the invention is directed to methods of decreasing triglyceride levels (e.g., in blood, plasma, or serum) in a mammal. The methods comprise administering a compound to the mammal, where administering the compound to the mammal decreases serum triglyceride levels in the mammal and

(a) directly causes an increase in an amino acid in the hypothalamus of the mammal, or

(b) causes an increase in metabolism of the amino acid or an α-keto acid corresponding to the amino acid toward acetyl-CoA in the hypothalamus of the mammal. In these embodiments, the compound is not pyruvate.

The invention is further directed to additional methods of decreasing triglyceride levels (e.g., in blood, plasma, or serum) in a mammal. The methods comprise administering a compound to the mammal in an amount effective to decrease serum triglyceride levels in the mammal, where the compound is an amino acid, an amino acid analog, an immediate precursor to an amino acid, an amino acid-increasing molecule, a nucleic acid encoding an amino acid-increasing molecule, a molecule that metabolizes an amino acid toward acetyl-CoA, a nucleic acid encoding a molecule that metabolizes an amino acid toward acetyl-CoA, or an inhibitor of an enzyme that decreases the activity of a molecule that metabolizes the amino acid toward acetyl-CoA.

The invention is additionally directed to methods of decreasing very low density lipoprotein (VLDL) levels (e.g., in blood, plasma, or serum) in a mammal. The methods comprise administering a compound to the mammal, where administering the compound to the mammal decreases VLDL levels in the mammal and

(a) directly causes an increase in an amino acid in the hypothalamus of the mammal, or

(b) causes an increase in metabolism of the amino acid or an α-keto acid corresponding to the amino acid toward acetyl-CoA in the hypothalamus of the mammal. In these embodiments, the compound is not pyruvate.

In further embodiments, the invention is directed to additional methods of decreasing very low density lipoprotein (VLDL) levels (e.g., in blood, plasma, or serum) in a mammal. The methods comprise administering a compound to the mammal in an amount effective to decrease VLDL levels in the mammal, where the compound is an amino acid, an amino acid analog, an immediate precursor to an amino acid, an amino acid-increasing molecule, a nucleic acid encoding an amino acid-increasing molecule, a molecule that metabolizes an amino acid toward acetyl-CoA, a nucleic acid encoding a molecule that metabolizes an amino acid toward acetyl-CoA, or an inhibitor of an enzyme that decreases the activity of a molecule that metabolizes the amino acid toward acetyl-CoA.

The invention also provides methods of treating a disorder in a mammal, the disorder selected from the group consisting of obesity, type 2 diabetes, type 1 diabetes, hyperglycemia, insulin resistance, glucose intolerance, leptin resistance, metabolic syndrome, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, and any combination of the foregoing by administering a compound of the invention (or a pharmaceutically acceptable salt or prodrug thereof) to the mammalian subject in a treatment effective amount.

The invention further provides a method of treating a disorder in a mammalian subject, the disorder selected from the group consisting of obesity, type 2 diabetes, type 1 diabetes, hyperglycemia, insulin resistance, glucose intolerance, leptin resistance, metabolic syndrome, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, gonadotropin deficiency, amenorrhea, lactic acidosis, polycystic ovary syndrome, and any combination thereof, the method comprising administering a compound to the mammal in a treatment-effective amount, wherein the compound

(a) directly causes an increase in an amino acid in the hypothalamus of the mammal, or

(b) causes an increase in metabolism of the amino acid or an α-keto acid corresponding to the amino acid toward acetyl-CoA in the hypothalamus of the mammal,

and further wherein the compound is not pyruvate.

As yet another aspect, the invention provides a method of treating a disorder in a mammalian subject, the disorder selected from the group consisting of obesity, type 2 diabetes, type 1 diabetes, hyperglycemia, insulin resistance, glucose intolerance, leptin resistance, metabolic syndrome, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, gonadotropin deficiency, amenorrhea, lactic acidosis, polycystic ovary syndrome, and any combination thereof, the method comprising administering a compound to the mammal in a treatment-effective amount, wherein the compound is an amino acid, an immediate precursor to an amino acid, an amino acid analog, an amino acid-increasing molecule, a nucleic acid encoding an amino acid-increasing molecule, a molecule that metabolizes an amino acid toward acetyl-CoA, a nucleic acid encoding a molecule that metabolizes an amino acid toward acetyl-CoA, or an inhibitor of an enzyme that decreases the activity of a molecule that metabolizes the amino acid toward acetyl-CoA.

In additional embodiments, the invention is directed to methods of increasing glucose production in a mammal. The methods comprise administering a compound to the mammal, where administering the compound to the mammal increases glucose production in the mammal and

(a) causes an decrease in an amino acid in the hypothalamus of the mammal provided the decrease is not due to metabolism of the amino acid toward acetyl-CoA, or

(b) causes an decrease in metabolism of the amino acid or an α-keto acid corresponding to the amino acid toward acetyl-CoA in the hypothalamus of the mammal.

The present invention is also directed to methods of increasing food intake in a mammal. The methods comprise administering a compound to the mammal, where administering the compound to the mammal increases food intake in the mammal and

(a) causes an decrease in an amino acid in the hypothalamus of the mammal provided the decrease is not due to metabolism of the amino acid toward acetyl-CoA, or

(b) causes an decrease in metabolism of the amino acid or an α-keto acid corresponding to the amino acid toward acetyl-CoA in the hypothalamus of the mammal.

In particular embodiments of the foregoing methods, the compound is administered intranasally, e.g., to deliver the compound to the CNS (including the brain) or the hypothalamus.

As yet another aspect, the invention provides a method of operating an intranasal delivery device comprising a compound of the invention or pharmaceutically acceptable salt or prodrug thereof. In representative embodiments, the invention provides a method of operating an intranasal delivery device comprising a pharmaceutical composition formulated for intranasal delivery, the pharmaceutical composition comprising a compound of the invention or pharmaceutically acceptable salt or prodrug thereof in a pharmaceutically acceptable carrier. Optionally, the device is configured and/or operated and/or the composition is formulated to enhance delivery to the upper third of the nasal cavity, superior meatus; olfactory region and/or the sinus region of the nose.

In particular embodiments, the invention provides a method of operating an intranasal delivery device, comprising: activating the intranasal delivery device to deliver a compound of the invention or pharmaceutically acceptable salt or prodrug thereof to a target location so that the compound or pharmaceutically acceptable salt or prodrug thereof is delivered to the CNS. Optionally, the compound is delivered as part of a pharmaceutical composition formulated for intranasal delivery. Optionally, the device is configured and/or operated and/or the composition is formulated to enhance delivery to the upper third of the nasal cavity, superior meatus, olfactory region and/or the sinus region of the nose along the olfactory neural pathway that has both intraneuronal and extraneuronal routes into the brain (Frey et al., (2002)Drug Delivery Tech. 2 (5): 46-49). According to this aspect of the invention, the activating step can further comprise positioning a unit dose container releasably holding the compound, pharmaceutically acceptable salt or prodrug thereof; nebulizing or atomizing the agent in the device; and releasing the nebulized or atomized agent intranasally.

Also provided is the use of a compound or pharmaceutical composition of the invention for reducing or increasing peripheral glucose levels (e.g., in blood, plasma or serum) of a mammal, reducing or increasing glucose production, inhibiting or increasing gluconeogenesis in the liver of a mammal, reducing food intake in a mammal, decreasing triglyceride levels (e.g., in blood, plasma or serum) in a mammal, decreasing VLDL levels in a mammal (e.g., in blood, plasma or serum), and/or for treating diabetes, metabolic syndrome, hyperglycemia, insulin resistance, glucose intolerance, leptin resistance, gonadotropin deficiency, amenorrhea, polycystic ovary syndrome, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, obesity and/or hypoglycemia in a mammal.

These and other aspects of the invention are set forth in more detail below.

### BRIEF DESCRIPTION OF THE DRAWINGS

FIG. 1 is an illustration of some relevant metabolic pathways in astrocytes and neurons.

FIG. 2 is a graph of experimental results providing evidence that a reduction in peripheral glucose release by central administration of proline.

FIG. 3 shows graphs of experimental results providing evidence that the reduction in peripheral glucose release by central administration of proline is due to reduced glucose production.

FIG. 4 shows graphs of experimental results providing evidence that metabolic flux through LDH is required for the central effects of proline on glucose production.

FIG. 5 shows graphs of experimental results providing evidence that administration of glutamine within the mediobasal hypothalamus is per se sufficient to inhibit glucose production.

FIG. 6 shows graphs of experimental results providing evidence that central administration of α-ketoisocaproic acid inhibits glucose production.

FIG. 7 shows graphs of experimental results providing evidence that administration of α-ketoisocaproic acid within the mediobasal hypothalamus is sufficient to suppress glucose production.

FIG. 8 shows graphs of experimental results providing evidence that metabolic flux through LDH is not required for the central effects of α-ketoisocaproic acid on glucose production.

FIG. 9 shows graphs of experimental results providing evidence that activation of K_{ATP} channels is required for the central effects of α-ketoisocaproic acid on glucose production.

### DETAILED DESCRIPTION OF THE INVENTION

The present invention is based in part on the inventors' discovery that increasing amino acid levels in the mammalian hypothalamus, or increasing amino acid metabolism toward acetyl-CoA in the hypothalamus, causes a reduction in glucose production, food intake, VLDL levels, and triglyceride levels in the mammal. See Example. Without being bound to any particular mechanism, it is believed that the amino acid signaling and/or metabolism causes the reduction in glucose production, food intake, VLDL levels, and triglyceride levels by increasing the hypothalamic nutrient signal induced by an increase in the rate of tricarboxylic acid (TCA) cycle flux through acetyl-CoA in the hypothalamus. This signal is described in PCT Patent Application No. PCT/US06/16967, filed May 2, 2006, incorporated by reference.

The present invention will now be described with reference to the accompanying drawings, in which preferred embodiments of the invention are shown. This invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have thesame meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

In some embodiments, the present invention is directed to methods of reducing peripheral glucose levels (e.g., in blood, plasma or serum) in a mammal. The methods comprise administering a compound to the mammal, where administering the compound to the mammal reduces peripheral glucose levels in the mammal and

(a) directly causes an increase in an amino acid in the hypothalamus of the mammal, or

(b) causes an increase in metabolism of the amino acid or an α-keto acid corresponding to the amino acid toward acetyl-CoA in the hypothalamus of the mammal. In these embodiments, the compound is not pyruvate.

In other embodiments, the invention is directed to additional methods of reducing peripheral glucose levels (e.g., in blood, plasma or serum) in a mammal. The methods comprise administering a compound to the mammal in an amount effective to reduce peripheral glucose levels in the mammal. In these embodiments, the compound is an amino acid, an immediate precursor to an amino acid, an amino acid analog, an amino acid-increasing molecule, a nucleic acid encoding an amino acid-increasing molecule, a molecule that metabolizes an amino acid toward acetyl-CoA, a nucleic acid encoding a molecule that metabolizes an amino acid toward acetyl-CoA, or an inhibitor of an enzyme that decreases the activity of a molecule that metabolizes the amino acid toward acetyl-CoA.

Glucose levels can be measured by any means known in the art. As used herein, “reducing peripheral glucose levels” (e.g., in blood, plasma or serum) and similar terms refer to a statistically significant reduction. The reduction can be, for example, at least about a 5%, 10%, 15%, 20%, 25%, 30%, 35%, 40%, 45%, 50%, 60%, 75% reduction or more.

Additionally, the invention is directed to methods of reducing food intake in a mammal. The methods comprise administering a compound to the mammal, where administering the compound to the mammal reduces food intake in the mammal and

(a) directly causes an increase in an amino acid in the hypothalamus of the mammal, or

(b) causes an increase in metabolism of the amino acid or an α-keto acid corresponding to the amino acid toward acetyl-CoA in the hypothalamus of the mammal. In these embodiments, the compound is not pyruvate.

The present invention is further directed to additional methods of reducing food intake in a mammal. The methods comprise administering a compound to the mammal in an amount effective to reduce food intake in the mammal, where the compound is an amino acid, an amino acid analog, an immediate precursor to an amino acid, an amino acid-increasing molecule, a nucleic acid encoding an amino acid-increasing molecule, a molecule that metabolizes an amino acid toward acetyl-CoA, a nucleic acid encoding a molecule that metabolizes an amino acid toward acetyl-CoA, or an inhibitor of an enzyme that decreases the activity of a molecule that metabolizes the amino acid toward acetyl-CoA.

In further embodiments, the invention is directed to methods of reducing glucose production in a mammal. The methods comprise administering a compound to the mammal, where administering the compound to the mammal reduces glucose production in the mammal and

(a) directly causes an increase in an amino acid in the hypothalamus of the mammal, or

(b) causes an increase in metabolism of the amino acid or an α-keto acid corresponding to the amino acid toward acetyl-CoA in the hypothalamus of the mammal. In these embodiments, the compound is not pyruvate.

Additionally, the invention is directed to additional methods of reducing glucose production in a mammal. The methods comprise administering a compound to the mammal in an amount effective to reduce glucose production in the mammal, where the compound is an amino acid, an amino acid analog, an immediate precursor to an amino acid, an amino acid-increasing molecule, a nucleic acid encoding an amino acid-increasing molecule, a molecule that metabolizes an amino acid toward acetyl-CoA, a nucleic acid encoding a molecule that metabolizes an amino acid toward acetyl-CoA, or an inhibitor of an enzyme that decreases the activity of a molecule that metabolizes the amino acid toward acetyl-CoA.

As used herein, the term “glucose production” can refer to whole animal glucose production, peripheral glucose production, or glucose production by particular organs or tissues (e.g., the liver and/or skeletal muscle). Glucose production can be determined by any method known in the art, e.g., by the pancreatic/insulin clamp technique.

In representative embodiments, glucose production is reduced by at least about 5%, 10%, 15%, 20%, 25%, 30%, 35%, 40%, 45%, 50%, 60%, 75% or more. In particular embodiments, glucose production is normalized (e.g., as compared with a suitable control) in the subject.

In additional embodiments, the invention is directed to methods of inhibiting gluconeogenesis in the liver of a mammal. The methods comprise administering a compound to the mammal, where administering the compound to the mammal inhibits gluconeogenesis in the liver of the mammal and

(a) directly causes an increase in an amino acid in the hypothalamus of the mammal, or

(b) causes an increase in metabolism of the amino acid or an α-keto acid corresponding to the amino acid toward acetyl-CoA in the hypothalamus of the mammal. In these embodiments, the compound is not pyruvate.

The invention further provides a method of inhibiting gluconeogenesis in the liver of a mammal, the method comprising administering a compound to the mammal in an amount effective to inhibit gluconeogenesis in the liver of the mammal, wherein the compound is an amino acid, an amino acid analog, an immediate precursor to an amino acid, an amino acid-increasing molecule, a nucleic acid encoding an amino acid-increasing molecule, a molecule that metabolizes an amino acid toward acetyl-CoA, a nucleic acid encoding a molecule that metabolizes an amino acid toward acetyl-CoA, or an inhibitor of an enzyme that decreases the activity of a molecule that metabolizes the amino acid toward acetyl-CoA.

Optionally, the reduction can be at least about a 5%, 10%, 15%, 20%, 25%, 30%, 35%, 40%, 45%, 50%, 60%, 75% reduction or more. Gluconeogenesis can be measured by any means known in the art, e.g., as described herein.

In other embodiments, the invention is directed to methods of decreasing triglyceride levels in a mammal (e.g., blood, serum or plasma triglyceride levels). The methods comprise administering a compound to the mammal, where administering the compound to the mammal decreases triglyceride levels in the mammal and

(a) directly causes an increase in an amino acid in the hypothalamus of the mammal, or

(b) causes an increase in metabolism of the amino acid or an α-keto acid corresponding to the amino acid toward acetyl-CoA in the hypothalamus of the mammal. In these embodiments, the compound is not pyruvate.

The invention is further directed to additional methods of decreasing triglyceride levels in a mammal (e.g., blood, serum or plasma triglyceride levels). The methods comprise administering a compound to the mammal in an amount effective to decrease triglyceride levels in the mammal, where the compound is an amino acid, an immediate precursor to an amino acid, an amino acid analog, an amino acid-increasing molecule, a nucleic acid encoding an amino acid-increasing molecule, a molecule that metabolizes an amino acid toward acetyl-CoA, nucleic acid encoding a molecule that metabolizes an amino acid toward acetyl-CoA, or an inhibitor of an enzyme that decreases the activity of a molecule that metabolizes the amino acid toward acetyl-CoA.

Triglyceride levels (e.g., in blood, plasma or serum) can be determined by any method known in the art.

In representative embodiments, triglyceride levels (e.g., in blood, plasma or serum) are reduced by at least about 5%, 10%, 15%, 20%, 25%, 30%, 35%, 40%, 45%, 50%, 60%, 75% or more. In particular embodiments, triglyceride levels are normalized (e.g., as compared with a suitable control) in the subject. Elevated and normal ranges of triglycerides can be readily determined. In particular embodiments, normal levels of serum triglycerides are in the range of 70-150 mg/dl.

The invention is additionally directed to methods of decreasing VLDL levels in a mammal (e.g., blood, plasma or serum VLDL levels). The methods comprise administering a compound to the mammal, where administering the compound to the mammal decreases VLDL levels in the mammal and

(a) directly causes an increase in an amino acid in the hypothalamus of the mammal, or

(b) causes an increase in metabolism of the amino acid or an α-keto acid corresponding to the amino acid toward acetyl-CoA in the hypothalamus of the mammal. In these embodiments, the compound is not pyruvate.

In further embodiments, the invention is directed to additional methods of decreasing VLDL levels in a mammal (e.g., blood, plasma or serum VLDL levels). The methods comprise administering a compound to the mammal in an amount effective to decrease VLDL levels in the mammal, where the compound is an amino acid, an immediate precursor to an amino acid, an amino acid analog, an amino acid-increasing molecule, a nucleic acid encoding an amino acid-increasing molecule, a molecule that metabolizes an amino acid toward acetyl-CoA, a nucleic acid encoding a molecule that metabolizes an amino acid toward acetyl-CoA, or an inhibitor of an enzyme that decreases the activity of a molecule that metabolizes the amino acid toward acetyl-CoA.

VLDL levels (e.g., in blood, plasma or serum) can be determined by any method known in the art.

In representative embodiments, VLDL levels (e.g., in blood, plasma or serum) are reduced by at least about 5%, 10%, 15%, 20%, 25%, 30%, 35%, 40%, 45%, 50%, 60%, 75% or more. In particular embodiments, VLDL levels are normalized (e.g., as compared with a suitable control) in the subject. Elevated and normal ranges of VLDL can be readily determined. In particular embodiments, normal levels of serum VLDL are in the range of 20-40 mg/dl.

In preferred embodiments of the methods described above, the mammal has a condition that would likely be at least partially alleviated by the methods of the invention. As used herein, partial alleviation of a condition is achieved if at least a small proportion of a group of mammals having the condition and subjected to the invention methods experience at least a partial reduction in the symptoms or progression of the condition as a result of the methods. Nonlimiting examples of such conditions include obesity, type 2 diabetes, type 1 diabetes, hyperglycemia, insulin resistance, glucose intolerance, leptin resistance, metabolic syndrome, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, gonadotropin deficiency, amenorrhea, lactic acidosis (including congenital lactic acidosis), polycystic ovary syndrome, and any combination of the foregoing

The invention is not limited to any particular methods of increasing an amino acid or metabolism of the amino acid or an α-keto acid corresponding to the amino acid toward acetyl-CoA in the hypothalamus of the mammal. In some embodiments, the compound is the amino acid.

As used herein, an amino acid is a chemical compound having amino and carboxylate functional groups covalently bound to the same carbon. The amino acid can be a glucogenic or a ketogenic amino acid. A glucogenic amino acid is an amino acid that gives rise to a net production of pyruvate or TCA cycle intermediates; a ketogenic amino acid is an amino acid that gives rise to acetyl-CoA or acetoacetyl-CoA. Some amino acids, for example, tryptophan, threonine, isoleucine, and phenylalanine are both glucogenic and ketogenic. Nonlimiting examples of glucogenic amino acids that can be utilized in these embodiments are alanine, arginine, asparagine, aspartate, cysteine, glycine, histidine, methionine, proline, serine, threonine, glutamine, glutamate, valine, isoleucine, phenylalanine, tryptophan, tyrosine, N-formaminoglutamate, homoserine, arginosuccinate, cystathionine, citrulline, homocysteine, ornithine, cysteinesulfinate, S-adenosylmethionine, S-adenosylhomocysteine, glutamate γ-semialdehyde, and 2-amino-3-ketobutyrate. Preferably, the glucogenic amino acid is glutamate or proline. See Example. Nonlimiting examples of ketogenic amino acids useful for these embodiments is isoleucine, leucine, tryptophan, lysine, phenylalanine, tyrosine, aspartate β-semialdehyde, β-aspartylphosphate, saccharopine, α-aminoadipate δ-semialdehyde, or α-aminoadipate. Preferably, the ketogenic amino acid is leucine.

The compound can also be an analog of the amino acids described above. Numerous amino acid analogs are known in the art. In particular embodiments, the amino acid analog includes the D or L configuration of an amino acid having the following formula: —NH—CHR—CO— wherein R is an aliphatic group, a substituted aliphatic group, a benzyl group, a substituted benzyl group, an aromatic group or a substituted aromatic group and wherein R does not correspond to the side chain of a naturally-occurring amino acid. As used herein, aliphatic groups include straight chained, branched or cyclic C1-C8 hydrocarbons which are completely saturated, which contain one or two heteroatoms such as nitrogen, oxygen or sulfur and/or which contain one or more units of unsaturation. Aromatic groups include carbocyclic aromatic groups such as phenyl and naphthyl and heterocyclic aromatic groups such as imidazolyl, indolyl, thienyl, furanyl, pyridyl, pyranyl, oxazolyl, benzothienyl, benzofuranyl, quinolinyl, isoquinolinyl and acridinyl.

Suitable substituents on an aliphatic, aromatic or benzyl group include —OH, halogen (—Br, —Cl, —I and —F) —O (aliphatic, substituted aliphatic, benzyl, substituted benzyl, aryl or substituted aryl group), —CN, —NO₂, —COOH, —NH₂, —NH(aliphatic group, substituted aliphatic, benzyl, substituted benzyl, aryl or substituted aryl group), —N(aliphatic group, substituted aliphatic, benzyl, substituted benzyl, aryl or substituted aryl group)₂, —COO(aliphatic group, substituted aliphatic, benzyl, substituted benzyl, aryl or substituted aryl group), —CONH₂, —CONH(aliphatic, substituted aliphatic group, benzyl, substituted benzyl, aryl or substituted aryl group), —SH, —S(aliphatic, substituted aliphatic, benzyl, substituted benzyl, aromatic or substituted aromatic group) and —NH—C(═NH)—NH₂. A substituted benzylic or aromatic group can also have an aliphatic or substituted aliphatic group as a substituent. A substituted aliphatic group can also have a benzyl, substituted benzyl, aryl or substituted aryl group as a substituent. A substituted aliphatic, substituted aromatic or substituted benzyl group can have one or more substituents. Modifying an amino acid substituent can increase, for example, the lipophilicity or hydrophobicity of natural amino acids that are hydrophilic.

A number of suitable amino acids, amino acid analogs and salts thereof can be obtained commercially. Others can be synthesized by methods known in the art. Synthetic techniques are described, for example, in Green and Wuts, “Protecting Groups in Organic Synthesis”, John Wiley and Sons, Chapters 5 and 7, 1991.

The compound in these embodiments can also be an immediate precursor of the amino acid, for example α-ketoglutarate, indole, α-ketobutarate, L-histadinol, phenylpyruvate, 4-hydroxyphenylpyruvate, methyltetrahydrofolate, α-keto-β-methylvalerate, or α-ketoisovalerate.

Administration of the compound can also cause an increase in metabolism of an α-keto acid corresponding to the amino acid toward acetyl-CoA. Nonlimiting examples of such α-keto acids are α-ketoisocaproic acid, oxaloacetate, α-ketoglutarate, α-keto-β-methylvalerate, α-ketoisovalerate, α-ketobutarate, citrulline, and α-ketoadipate: In particular embodiment, the α-keto acid is administered.

In other embodiments, the compound increases the activity of an amino acid-increasing molecule in the hypothalamus of the mammal. Included herewith are the amino acid-increasing molecules and nucleic acid encoding the amino acid-increasing molecules. As used herein, “increasing the activity” encompasses methods that increase the action of a preexisting molecule, or increasing the amount of such molecules, or combinations thereof. The amount of a molecule can be increased by reducing the rate of degradation or removal of the molecule and/or increasing the biosynthesis of the molecule and/or adding the molecule. Conversely, “decreasing the activity” of a molecule means either reducing the action (e.g., enzyme activity) of a preexisting molecule, or reducing the amount of such molecules, or combinations thereof. It should be understood that the amount of the molecules can be reduced by increasing the rate of degradation or removal of the molecule and/or reducing the biosynthesis of the molecule.

Examples of amino acid-increasing molecules are glutamate dehydrogenases, valine aminotransferases, arginosuccinate lyases, tyrosine transaminases, aromatic amino acid transaminases, tryptophan synthases, and histidinol dehydrogenases. In particular embodiments, the amino acid increasing molecule or nucleic acid encoding the same is administered.

The compound can also increase the activity of a molecule that metabolizes the amino acid toward acetyl-CoA. Included herewith are the molecules that metabolize the amino acid toward acetyl-CoA, and nucleic acids encoding the molecules. Examples of such molecules are threonine dehydrogenases, tyrosine aminotransferases, serine dehydratases, acyl-CoA dehydrogenases, branched chain amino acid transaminases, and branched chain α-ketoacid dehydrogenases. In preferred embodiments, the molecule that metabolizes the amino acid toward acetyl-CoA is a branched chain α-ketoacid dehydrogenase. The compounds in these embodiments can inhibit an enzyme that decreases the activity of the molecule that metabolizes the amino acid toward acetyl-CoA, for example α-chloroisocaproic acid (α-CIC), an inhibitor of a kinase that phosphorylates and inhibits branched chain α-ketoacid dehydrogenase. See Example.

In some of these methods, the compound inhibits an enzyme. Additionally or alternatively, the compound inhibits a protein that regulates an enzyme, for example a kinase or a phosphatase. The compound can also inhibit a protein that inhibits metabolism of the amino acid toward acetyl-CoA. An example of a compound of this type is α-chloroisocaproic acid (α-CIC).

The invention also provides methods of treating a disorder in a mammal, the disorder selected from the group consisting of obesity, type 2 diabetes, type 1 diabetes, hyperglycemia, insulin resistance, glucose intolerance, leptin resistance, metabolic syndrome, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, gonadotropin deficiency, amenorrhea, lactic acidosis (including congenital lactic acidosis), polycystic ovary syndrome, and any combination of the foregoing by administering a compound of the invention as described above to the mammalian subject in a treatment effective amount.

In particular embodiments, the invention provides a method of treating a disorder in a mammalian subject, the disorder selected from the group consisting of obesity, type 2 diabetes, type 1 diabetes, hyperglycemia, insulin resistance, glucose intolerance, leptin resistance, metabolic syndrome, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, gonadotropin deficiency, amenorrhea, lactic acidosis (including congenital lactic acidosis), polycystic ovary syndrome, and any combination thereof, the method comprising administering a compound to the mammal in a treatment-effective amount, wherein the compound

(a) directly causes an increase in an amino acid in the hypothalamus of the mammal, or

(b) causes an increase in metabolism of the amino acid or an α-keto acid corresponding to the amino acid toward acetyl-CoA in the hypothalamus of the mammal,

and further wherein the compound is not pyruvate.

In other embodiments, the invention provides a method of treating a disorder in a mammalian subject, the disorder selected from the group consisting of obesity, type 2 diabetes, type 1 diabetes, hyperglycemia, insulin resistance, glucose intolerance, leptin resistance, metabolic syndrome, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, gonadotropin deficiency, amenorrhea, lactic acidosis (including congenital lactic acidosis), polycystic ovary syndrome, and any combination thereof, the method comprising administering a compound to the mammal in a treatment-effective amount, wherein the compound is an amino acid, an amino acid analog, an immediate precursor to an amino acid, an amino acid-increasing molecule, a nucleic acid encoding an amino acid-increasing molecule, a molecule that metabolizes an amino acid toward acetyl-CoA, a nucleic acid encoding a molecule that metabolizes an amino acid toward acetyl-CoA, or an inhibitor of an enzyme that decreases the activity of a molecule that metabolizes the amino acid toward acetyl-CoA.

As used herein, an “effective amount” refers to an amount of a compound or pharmaceutical composition that is sufficient to produce a desired effect, which is optionally a therapeutic effect (i.e., by administration of a treatment effective amount). For example, an “effective amount” can be an amount that is sufficient to reduce glucose production, reduce peripheral glucose levels, to reduce gluconeogenesis in the liver, to reduce triglyceride levels, to reduce VLDL levels, to treat metabolic disorders such as metabolic syndrome, hyperglycemia, glucose intolerance, insulin resistance, diabetes (e.g., type-1 or type-2 diabetes), and/or obesity and/or to treat leptin resistance, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, to treat gonadotropin deficiency, amenorrhea and/or polycystic ovary syndrome.

A “treatment effective” amount as used herein is an amount that provides some improvement or benefit to the subject. Alternatively stated, a “treatment effective” amount is an amount that provides some alleviation, mitigation, delay and/or decrease in at least one clinical symptom and/or prevent the onset or progression of at least one clinical symptom. Clinical symptoms associated with the disorders that can be treated by the methods of the invention are well-known to those skilled in the art. Further, those skilled in the art will appreciate that the therapeutic effects need not be complete or curative, as long as some benefit is provided to the subject.

By the terms “treat,” “treating” or “treatment of” (or grammatically equivalent terms) it is meant that the severity of the subject's condition is reduced or at least partially improved or ameliorated and/or that some alleviation, mitigation or decrease in at least one clinical symptom is achieved and/or there is a delay in the progression of the condition and/or prevention or delay of the onset of a disease or illness. Thus, the terms “treat,” “treating” or “treatment of” (or grammatically equivalent terms) refer to both prophylactic and therapeutic treatment regimes.

The foregoing methods are expected to be useful in conjunction with any other disease treatment or prophylaxis, including but not limited to administration of a second compound, where the second compound is useful for the prevention or treatment of obesity, type 2 diabetes, type 1 diabetes, hyperglycemia, insulin resistance, glucose intolerance, leptin resistance, metabolic syndrome, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, gonadotropin deficiency, amenorrhea, lactic acidosis (including congenital lactic acidosis) and/or polycystic ovary syndrome, and/or a complication thereof. Nonlimiting examples of such compounds are insulin, antihyperglycemic agents such as troglitazone or metformin, α-glucosidase inhibitors such as acarbose, rennin-angiotensin system blockers such as ramipril, antihypertensive drugs such as ACE inhibitors, angiotensin receptor blockers, β-blockers, diuretics, calcium channel blockers, anti-platelet agents such as clopidogrel, poly (ADP-ribose) polymerase (PARP) inhibitors such as PJ34, 3 aminobenzamide, 4 amino 1,8 naphthalimide, 6(5H) phenanthridinone, benzamide, INO 1001, and NU1025, and lipid management agents such as statins, ezetinbe, niacin, fibric acid derivatives, and bile acid sequestrants such as fenofibrate.

The skilled artisan understands that decreasing amino acids, or the metabolism of the amino acids or corresponding α-keto acids toward acetyl-CoA in the hypothalamus has the opposite effect as increasing those parameters, i.e., causes increases in glucose production and food intake.

Thus, in additional embodiments, the invention is directed to methods of increasing glucose production in a mammal. The methods comprise administering a compound to the mammal, where administering the compound to the mammal increases glucose production in the mammal and

(a) causes an decrease in an amino acid in the hypothalamus of the mammal provided the decrease is not due to metabolism of the amino acid toward acetyl-CoA, or

(b) causes an decrease in metabolism of the amino acid or an α-keto acid corresponding to the amino acid toward acetyl-CoA in the hypothalamus of the mammal.

These methods are useful in any situation where it is desired that the mammal increase its glucose production. Examples of such situations are when the mammal is undergoing a treatment that causes insufficient glucose production, for example cancer chemotherapy, or when the mammal has an infection, such as a viral infection (e.g., HIV-1 infection) that causes insufficient glucose production. The methods are also effective in mammals that are hypoglycemic.

These embodiments are not limited to administration of any particular compound or group of compounds that causes a decrease, or a decrease in metabolism, of an amino acid or corresponding α-keto acid. Examples of such compounds are those that increase the activity of an amino acid-decreasing molecule in the hypothalamus of the mammal, and those that decrease the activity of an amino acid-increasing molecule in the hypothalamus of the mammal. Nonlimiting examples of amino acid-increasing molecules are glutamate dehydrogenases, valine aminotransferases, arginosuccinate lyases, tyrosine transaminases, aromatic amino acid transaminases, tryptophan synthases, and histidinol dehydrogenases. In these aspects, preferred compounds are small organic molecule inhibitors (e.g., less than about 1000, 1500 or 2000 daltons) of amino acid-increasing molecules. One such compound is guanosine 5′-triphosphate (GTP). Another class of preferred compounds in these embodiments are antibodies and aptamers that specifically inhibit acetyl-CoA increasing molecules.

The present invention is also directed to methods of increasing food intake in a mammal. The methods comprise administering a compound to the mammal, where administering the compound to the mammal increases food intake in the mammal and

(a) causes an decrease in an amino acid in the hypothalamus of the mammal provided the decrease is not due to metabolism of the amino acid toward acetyl-CoA, or

(b) causes an decrease in metabolism of the amino acid or an α-keto acid corresponding to the amino acid toward acetyl-CoA in the hypothalamus of the mammal.

These methods are useful in any situation where it is desired that the mammal increase its food intake. Examples of such situations are when the mammal is undergoing a treatment that causes insufficient food intake, for example cancer chemotherapy, or when the mammal has an infection, such as a viral infection (e.g., HIV-1 infection) that causes insufficient food intake. The methods are also effective in mammals that are hypoglycemic.

These embodiments are not limited to administration of any particular compound or group of compounds that causes a decrease, or a decrease in metabolism, of an amino acid or corresponding α-keto acid. Examples of such compounds are those that increase the activity of an amino acid-decreasing molecule in the hypothalamus of the mammal, and those that decrease the activity of an amino acid-increasing molecule in the hypothalamus of the mammal. Nonlimiting examples of amino acid-increasing molecules are glutamate dehydrogenases, valine aminotransferases, arginosuccinate lyases, tyrosine transaminases, aromatic amino acid transaminases, tryptophan synthases, and histidinol dehydrogenases. In these aspects, preferred compounds are small organic molecule inhibitors (e.g., less than about 1000, 1500 or 2000 daltons) of amino acid-increasing molecules. One such compound is guanosine 5′-triphosphate (GTP). Another class of preferred compounds in these embodiments are antibodies and aptamers that specifically inhibit acetyl-CoA increasing molecules.

Those skilled in the art will appreciate that the active compounds of the invention as described above can optionally be in the form of pharmaceutically acceptable salts.

Further, the compounds of the invention include prodrugs that are converted to the active compound in vivo. For example, the compound can be modified to enhance cellular permeability (e.g., by esterification of polar groups) and then converted by cellular enzymes to produce the active agent. Methods of masking charged or reactive moieties as a pro-drug are known by those skilled in the art (see, e.g., P. Korgsgaard-Larsen and H. Bundgaard, A Textbook of Drug Design and Development, Reading U.K., Harwood Academic Publishers, 1991).

The term “prodrug” refers to compounds that are transformed in vivo to yield the parent compound of the above formula, for example, by hydrolysis in blood, see, e.g., T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299. Exemplary prodrugs include a prodrug that is metabolized in vivo by a subject to an active drug having an activity of the compounds as described herein, wherein the prodrug is an ester of an alcohol or carboxylic acid group, if such a group is present in the compound; an amide of an amine group or carboxylic acid group, if such groups are present in the compound; a urethane of an amine group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described, for example, in U.S. Pat. No. 6,680,324 and U.S. Pat. No. 6,680,322. Other prodrugs will be apparent to those skilled in the art.

The methods of the invention can be practiced with any species of mammal, including without limitation humans, non-human primates, mice, rats, rabbits, cattle, sheep, goats, pigs, horses, cats, dogs, including non-human mammals that are experimental models of human disease.

The subject can be a subject “in need of” the methods of the present invention, e.g., in need of the therapeutic and/or prophylactic effects of the inventive methods. For example, the subject can be one that has been diagnosed with or is considered at risk for diabetes (type 1 or type 2), metabolic syndrome, hyperglycemia, insulin resistance, glucose intolerance, obesity, leptin resistance, gonadotropin deficiency, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, amenorrhea, and/or polycystic ovary syndrome, and the methods and compositions of the invention are used for therapeutic and/or prophylactic treatment.

As discussed in more detail below, in particular embodiments, the compound is delivered to the CNS (e.g., brain) or hypothalamus. Delivery to the CNS or hypothalamus can be by any route including by peripheral or central administration routes. In particular embodiments, delivery to the CNS or hypothalamus is by an intranasal route of administration.

As used herein, the term “CNS” can refer to the CNS as a whole or to particular parts of the CNS, e.g., the brain, the hypothalamus, the mediobasal hypothalamus, the ARC and/or the vagus nerve. Likewise, as used herein, the terms “delivery to,” “administering to,” “administration to” the CNS (and similar terms) can refer to the CNS when assessed as a whole, or can refer to particular regions/tissues of the CNS.

As used herein, the term “hypothalamus” can refer to the hypothalamus as a whole or to particular regions of the hypothalamus (e.g., the mediobasal hypothalamus or the arcuate nucleus [ARC]). Likewise, as used herein, the terms “delivery to,” “administering to,” or “administration to” the hypothalamus (and similar terms) can refer to the hypothalamus when assessed as a whole, or can refer to particular regions of the hypothalamus (e.g., the mediobasal hypothalamus or the ARC).

The invention also provides pharmaceutical formulations comprising an active compound of the invention as described above (or a pharmaceutically acceptable prodrug or salt thereof) in a pharmaceutically acceptable excipient.

By “pharmaceutically acceptable” it is meant a material that (i) is compatible with the other ingredients of the composition without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers include, without limitation, any of the standard pharmaceutical carriers such as phosphate buffered saline solutions, water, emulsions such as oil/water emulsions, microemulsions, and the like.

The pharmaceutical compositions of the present invention can optionally be administered in conjunction with other therapeutic agents, for example, other therapeutic agents useful in the treatment of hyperglycemia, diabetes, metabolic syndrome hypertriglyceridemia, hyperVLDLemia and/or obesity. For example, the compounds of the invention can be administered in conjunction with insulin therapy and/or hypoglycemic agents (e.g., metformin). The additional therapeutic agent(s) can be administered concurrently with the compounds of the invention, in the same or different formulations. As used herein, the word “concurrently” means sufficiently close in time to produce a combined effect (that is, concurrently can be simultaneously, or it can be two or more events occurring within a short time period before or after each other).

The above-described compounds can be formulated without undue experimentation for administration to a mammal, including humans, as appropriate for the particular application. Additionally, proper dosages of the compositions can be determined without undue experimentation using standard dose-response protocols.

A treatment effective amount will vary with the age and general condition of the subject, the efficiency of the delivery method (i.e., the percent of the dose that is deposited in the target area), the severity of the condition being treated, the particular compound or composition being administered, the duration of the treatment, the nature of any concurrent treatment, the carrier used, and like factors within the knowledge and expertise of those skilled in the art. As appropriate, a treatment effective amount in any individual case can be determined by one of ordinary skill in the art by reference to the pertinent texts and literature and/or by using routine experimentation (see, e.g., Remington, The Science and Practice of Pharmacy (20^{th} ed. 2000)).

As a general proposition, a dosage from about 0.1 to about 5, 10, 20, 50, 75 or 100 mg active agent/kg body weight will have therapeutic efficacy, with all weights being calculated based upon the weight of the active ingredient, including salts.

Accordingly, compositions designed for oral, lingual, sublingual, buccal and intrabuccal administration can be made without undue experimentation by means well known in the art, for example with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, cornstarch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

The compounds can easily be administered parenterally such as for example, by intravenous, intramuscular, intrathecal or subcutaneous injection. Parenteral administration can be accomplished by incorporating the compounds into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the compound, in a pharmaceutical composition, into the rectum or large intestine. This can be accomplished using suppositories or enemas. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the composition through the skin. Transdermal formulations include patches (such as the well-known nicotine patch), ointments, creams, gels, salves and the like.

The compounds of the invention can be delivered to the CNS (e.g., brain) or more specifically, the hypothalamus. The blood-brain barrier presents a barrier to the passive diffusion of substances from the bloodstream into various regions of the CNS. However, active transport of certain agents is known to occur in either direction across the blood-brain barrier. Substances that may have limited access to the brain from the bloodstream can be injected directly into the cerebrospinal fluid. Cerebral ischemia and inflammation are also known to modify the blood-brain barrier and result in increased access to substances in the bloodstream.

Administration of a therapeutic compound directly to the brain is known in the art. Intrathecal injection administers agents directly to the brain ventricles and the spinal fluid. Surgically-implantable infusion pumps are available to provide sustained administration of agents directly into the spinal fluid. Lumbar puncture with injection of a pharmaceutical compound into the cerebrospinal fluid (“spinal injection”) is known in the art, and is suited for administration of compounds and compositions according to the present invention. In particular embodiments, intracerebroventricular (ICV) administration is used to deliver the compound (e.g., ICV injection through a surgically implanted cannulae). According to this embodiment, the ICV administration can be to the third cerebral ventricle of the brain. Thus, in representative embodiments, the active compound can be administered directly to the brain of the mammal, e.g., by direct injection or through a pump.

Alternatively, the active compound of the invention can be administered peripherally in a manner that permits the activator to cross the blood-brain barrier of the mammal.

For example, the active compound can be formulated in a pharmaceutical composition that enhances the ability of the compound(s) to cross the blood-brain barrier of the mammal. Pharmacologic-based procedures are known in the art for circumventing the blood brain barrier, including the conversion of hydrophilic compounds into lipid-soluble drugs. For example, the active compound can be encapsulated in a lipid vesicle or liposome.

Further, the active compound can be administered by a method that results in the compound crossing the blood-brain barrier. For example, the intra-arterial infusion of hypertonic substances to transiently open the blood-brain barrier and allow passage of hydrophilic drugs into the brain is also known in the art. U.S. Pat. No. 5,686,416 to Kozarich et al. discloses the co-administration of receptor mediated permeabilizer (RMP) peptides with therapeutic compounds to be delivered to the interstitial fluid compartment of the brain, to cause an increase in the permeability of the blood-brain barrier and effect increased delivery of the therapeutic compounds to the brain. Intravenous or intraperitoneal administration may also be used in practicing the present invention.

One method of transporting an active agent across the blood-brain barrier is to couple or conjugate the active compound to a second molecule (a “carrier”), which is a peptide or non-proteinaceous moiety selected for its ability to penetrate the blood-brain barrier and transport the active agent across the blood-brain barrier. Examples of suitable carriers include pyridinium, fatty acids, inositol, cholesterol, and glucose derivatives. The carrier may be a compound that enters the brain through a specific transport system in brain endothelial cells. Chimeric peptides adapted for delivering neuropharmaceutical agents into the brain by receptor-mediated transcytosis through the blood-brain barrier are disclosed in U.S. Pat. No. 4,902,505 to Pardridge et al. These chimeric peptides comprise a pharmaceutical agent conjugated with a transportable peptide capable of crossing the blood-brain barrier by transcytosis. Specific transportable peptides disclosed by Pardridge et al. include histone, insulin, transferrin, and others. Conjugates of a compound with a carrier molecule, to cross the blood-brain barrier, are also disclosed in U.S. Pat. No. 5,604,198 to Poduslo et al. Specific carrier molecules disclosed include hemoglobin, lysozyme, cytochrome c, ceruloplasmin, calmodulin, ubiquitin and substance P. See also U.S. Pat. No. 5,017,566 to Bodor.

Where the active compound of the invention is administered peripherally such that it crosses the blood-brain barrier, the active compound can be formulated in a pharmaceutical composition that enhances the ability of the compound to cross the blood-brain barrier of the mammal. Such formulations are known in the art and include lipophilic compounds to promote absorption. Uptake of non-lipophilic compounds can be enhanced by combination with a lipophilic substance. Lipophilic substances that can enhance delivery of the compound across the nasal mucus include but are not limited to fatty acids (e.g., palmitic acid), gangliosides (e.g., GM-1), phospholipids (e.g., phosphatidylserine), and emulsifiers (e.g., polysorbate 80), bile salts such as sodium deoxycholate, and detergent-like substances including, for example, polysorbate 80 such as Tween™, octoxynol such as Triton™ X-100, and sodium tauro-24,25-dihydrofusidate (STDHF). See Lee et al., Biopharm., April 1988 issue:3037.

In particular embodiments of the invention, the active compound is combined with micelles comprised of lipophilic substances. Such micelles can modify the permeability of the nasal membrane to enhance absorption of the compound. Suitable lipophilic micelles include without limitation gangliosides (e.g., GM-1 ganglioside), and phospholipids (e.g., phosphatidylserine). Bile salts and their derivatives and detergent-like substances can also be included in the micelle formulation. The active compound can be combined with one or several types of micelles, and can further be contained within the micelles or associated with their surface.

Alternatively, the active compound can be combined with liposomes (lipid vesicles) to enhance absorption. The active compound can be contained or dissolved within the liposome and/or associated with its surface. Suitable liposomes include phospholipids (e.g., phosphatidylserine) and/or gangliosides (e.g., GM-1). For methods to make phospholipid vesicles, see for example, U.S. Pat. No. 4,921,706 to Roberts et al., and U.S. Pat. No. 4,895,452 to Yiournas et al. Bile salts and their derivatives and detergent-like substances can also be included in the liposome formulation.

Other methods of delivering compounds across the blood-brain barrier are well-known in the art. Methods of intranasal and pulmonary delivery (e.g., to the CNS, more specifically, the hypothalamus) are discussed in more detail below.

### Pharmaceutical Formulations and Modes of Intranasal Delivery.

The invention also encompasses pharmaceutical compositions formulated for intranasal administration comprising one or more active compounds of the invention or pharmaceutically acceptable salts or prodrugs thereof in a pharmaceutically acceptable excipient.

The compositions and methods of the present invention provide for the delivery of compounds to the CNS by the nasal route for example, by the olfactory neural pathway (Frey et al., (2002) Drug Delivery Tech. 2 (5): 46-49), while minimizing systemic exposure. In this regard and without being bound by any particular theory, it is believed that targeting the CNS by nasal administration is based on capture and internalization of substances by the olfactory receptor neurons, which substances are then transported inside the neuron to the olfactory bulb of the brain. Olfactory receptor neurons from the lateral olfactory tract within the olfactory bulb project to various regions such as the hippocampus, amygdala, thalamus, hypothalamus and other regions of the brain that are not directly involved in olfaction. These substances may also pass through junctions in the olfactory epithelium at the olfactory bulb and enter the subarachnoid space, which surrounds the brain, and the cerebral spinal fluid (CSF), which bathes the brain. Either pathway allows for targeted delivery without interference by the blood brain barrier, as neurons and the CSF, not the circulatory system, are involved in these transport mechanisms. Accordingly, intranasal delivery pathways permit compartmentalized delivery of compounds with substantially reduced systemic exposure and the resulting side effects.

As further advantages, nasal delivery offers a noninvasive means of administration that is generally safe and convenient for self-medication. Intranasal administration can also provide for rapid onset of action due to ready absorption by the nasal mucosa. This characteristic of nasal delivery results from several factors, including: (1) the nasal cavity has a relatively large surface area of about 150 cm² in humans, (2) the submucosa of the lateral wall of the nasal cavity is richly supplied with vasculature, and (3) the nasal epithelium provides for a relatively high drug permeation capability due to thin single cellular layer absorption.

The formulations of the invention can optionally comprise medicinal agents, pharmaceutical agents, carriers, dispersing agents, diluents, humectants, wetting agents, thickening agents, odorants, humectants, penetration enhancers, preservatives, and the like.

The compositions of the invention can be formulated for intranasal administration in a pharmaceutical carrier in accordance with known techniques. See, e.g., Remington, The Science And Practice of Pharmacy (20^{th} edition, 2000). Suitable nontoxic pharmaceutically acceptable nasal carriers will be apparent to those skilled in the art of nasal pharmaceutical formulations (see, e.g., Remington's Pharmaceutical Sciences (Maack Publishing Co., Easton latest edition). Further, it will be understood by those skilled in the art that the choice of suitable carriers, absorption enhancers, humectants, adhesives, etc., will typically depend on the nature of the active compound and the particular nasal formulation, for example, a nasal solution (e.g., for use as drops, spray or aerosol), a nasal suspension, a nasal ointment, a nasal gel, or another nasal formulation.

The carrier can be a solid or a liquid, or both, and is optionally formulated with the composition as a unit-dose formulation. Such dosage forms can be powders, solutions, suspensions, emulsions and/or gels. With respect to solutions or suspensions, dosage forms can be comprised of micelles of lipophilic substances, liposomes (phospholipid vesicles/membranes) and/or a fatty acid (e.g., palmitic acid). In particular embodiments, the pharmaceutical composition is a solution or suspension that is capable of dissolving in the fluid secreted by mucous membranes of the epithelium of the nasal cavity, which can advantageously enhance absorption.

The pharmaceutical composition can be an aqueous solution, a nonaqueous solution or a combination of an aqueous and nonaqueous solution.

Suitable aqueous solutions include but are not limited to aqueous gels, aqueous suspensions, aqueous microsphere suspensions, aqueous microsphere dispersions, aqueous liposomal dispersions, aqueous micelles of liposomes, aqueous microemulsions, and any combination of the foregoing, or any other aqueous solution that can dissolve in the fluid secreted by the mucosal membranes of the nasal cavity. Exemplary nonaqueous solutions include but are not limited to nonaqueous gels, nonaqueous suspensions, nonaqueous microsphere suspensions, nonaqueous microsphere dispersions, nonaqueous liposomal dispersions, nonaqueous emulsions, nonaqueous microemulsions, and any combination of the foregoing, or any other nonaqueous solution that can dissolve or mix in the fluid secreted by the mucosal membranes of the nasal cavity.

Examples of powder formulations include without limitation simple powder mixtures, micronized powders, powder microspheres, coated powder microspheres, liposomal dispersions, and any combination of the foregoing. Powder microspheres can be formed from various polysaccharides and celluloses, which include without limitation starch, methylcellulose, xanthan gum, carboxymethylcellulose, hydroxypropyl cellulose, carbomer, alginate polyvinyl alcohol, acacia, chitosans, and any combination thereof.

In particular embodiments, the compound is one that is at least partially, or even substantially (e.g., at least 80%, 90%, 95% or more) soluble in the fluids that are secreted by the nasal mucosa (e.g., the mucosal membranes that surround the cilia of the olfactory receptor cells of the olfactory epithelium) so as to facilitate absorption. Alternatively or additionally, the compound can be formulated with a carrier and/or other substances that foster dissolution of the agent within nasal secretions, including without limitation fatty acids (e.g., palmitic acid), gangliosides (e.g., GM-1), phospholipids (e.g., phosphatidylserine), and emulsifiers (e.g., polysorbate 80).

Optionally, drug solubilizers can be included in the pharmaceutical composition to improve the solubility of the compound and/or to reduce the likelihood of disruption of nasal membranes which can be caused by application of other substances, for example, lipophilic odorants. Suitable solubilizers include but are not limited to amorphous mixtures of cyclodextrin derivatives such as hydroxypropylcylodextrins (see, for example, Pitha et al., (1988) Life Sciences 43:493-502).

In representative embodiments, the compound is lipophilic to promote absorption. Optionally, uptake of non-lipophilic compounds can be enhanced by combination with a lipophilic substance. Lipophilic substances that can enhance delivery of the compound across the nasal mucosa include but are not limited to esters, fatty acids (e.g., palmitic acid), gangliosides (e.g., GM-1), phospholipids (e.g., phosphatidylserine), and emulsifiers (e.g., polysorbate 80), bile salts such as sodium deoxycholate, and detergent-like substances including, for example, polysorbate 80 such as Tween™, octoxynol such as Triton™ X-100, and sodium tauro-24,25-dihydrofusidate (STDHF) (see Lee et al., Biopharm., April 1988 issue:3037).

In particular embodiments of the invention, the active compound is combined with micelles comprised of lipophilic substances. Such micelles can modify the permeability of the nasal membrane to enhance absorption of the compound. Suitable lipophilic micelles include without limitation gangliosides (e.g., GM-1 ganglioside), and phospholipids (e.g., phosphatidylserine). Bile salts and their derivatives and detergent-like substances can also be included in the micelle formulation. The active compound can be combined with one or several types of micelles, and can further be contained within the micelles or associated with their surface.

Alternatively, the active compound can be combined with liposomes (lipid vesicles) to enhance absorption. The active compound can be contained or dissolved within the liposome and/or associated with its surface. Suitable liposomes include phospholipids (e.g., phosphatidylserine) and/or gangliosides (e.g., GM-1). For methods to make phospholipid vesicles, see for example, U.S. Pat. No. 4,921,706 to Roberts et al., and U.S. Pat. No. 4,895,452 to Yiournas et al. Bile salts and their derivatives and detergent-like substances can also be included in the liposome formulation.

In representative embodiments, the pH of the pharmaceutical composition ranges from about 2, 3, 3.5 or 5 to about 7, 8 or 10. Exemplary pH ranges include without limitation from about 2 to 8, from about 3.5 to 7, and from about 5 to 7. Those skilled in the art will appreciate that because the volume of the pharmaceutical composition administered is generally small, nasal secretions may alter the pH of the administered dose since the range of pH in the nasal cavity can be as wide as 5 to 8. Such alterations can affect the concentration of un-ionized drug available for absorption. Accordingly, in representative embodiments, the pharmaceutical composition further comprises a buffer to maintain or regulate pH in situ. Typical buffers include but are not limited to acetate, citrate, prolamine, carbonate and phosphate buffers.

In embodiments of the invention, the pH of the pharmaceutical composition is selected so that the internal environment of the nasal cavity after administration is on the acidic to neutral side, which (1) can provide the active compound in an un-ionized form for absorption, (2) prevents growth of pathogenic bacteria in the nasal passage, which is more likely to occur in an alkaline environment, and (3) reduces the likelihood of irritation of the nasal mucosa.

Further, in particular embodiments, the net charge on the compound is a positive or neutral charge.

For liquid and powder sprays or aerosols, the pharmaceutical composition can be formulated to have any suitable and desired particle or droplet size. In illustrative embodiments, the majority and/or the mean size of the particles or droplets range from equal to or greater than about 1, 2.5, 5, 10, 12, 15 or 20 microns and/or equal to or less than about 15, 18, 20, 25, 30, 40, 50, 60 or 75 microns (including all combinations of the foregoing as long as the lower end of the range is smaller than the upper end of the range). Representative examples of suitable ranges for the majority and/or mean particle or droplet size include, without limitation, from about 5 to 50 microns, from about 20 to 50 microns, from about 15 to 30 microns, from about 10 to 18 microns, from about 10 to 15 microns, and from about 12 to 15 microns which facilitate the deposition of an effective amount of the active compound in the nasal cavity (e.g., in the upper third of the nasal cavity, the superior meatus, the olfactory region and/or the sinus region to target the olfactory neural pathway). In general, particles or droplets smaller than about 5 microns will be deposited in the trachea or even the lung, whereas particles or droplets that are about 50 microns or larger generally do not reach the nasal cavity and are deposited in the anterior nose.

International patent publication WO 2005/023335 (Kurve Technology, Inc.) describes particles and droplets having a diameter size suitable for the practice of representative embodiments of the present invention. For example, the particles or droplets can have a mean diameter of about 2 to 50 microns, about 5 to 50 microns, about 5 to 40 microns, about 5 to 35 microns, about 5 to 30 microns, about 5 to 20 microns, about 5 to 17 microns, about 5 to 30 microns, about 10 to 25 microns, about 10 to 15 microns, about 11 to 50 microns, about 11 to 30 microns, about 11 to 20 microns, about 11 to 15 microns, about 12 to 17 microns, about 15 to 25 microns, about 15 to 27 microns or about 17 to 23 microns.

In particular embodiments, the particles or droplets have a mean diameter of about 5 to 30 microns, about 10 to 20 microns, about 10 to 17 microns, about 10 to 15 microns, about 12 to 17 microns, about 10 to 15 microns, about 10 to 12 microns or about 12 to 15 microns.

Further, the particles or droplets can have a mean diameter of about 10 to 20 microns, about 10 to 25 microns, about 10 to 30 microns, or about 15 to 30 microns.

The particles can “substantially” have a mean diameter or size as described herein, i.e., at least about 50%, 60%, 70%, 80%, 90% or 95 or more of the particles are of the indicated diameter or size range.

The composition is optionally delivered as a nebulized or atomized liquid having a droplet size as described above.

In particular embodiments, the pharmaceutical composition is isotonic to slightly hypertonic, e.g., having an osmolarity ranging from about 150 to 550 mOsM. As another particular example, the pharmaceutical composition is isotonic having, e.g., an osmolarity ranging from approximately 150 to 350 mOsM.

According to particular methods of intranasal delivery, it can be desirable to prolong the residence time of the pharmaceutical composition in the nasal cavity (e.g., in the upper third of the nasal cavity, the superior meatus, the olfactory region and/or in the sinus region), for example, to enhance absorption. Thus, the pharmaceutical composition can optionally be formulated with a bioadhesive polymer, a gum (e.g., xanthan gum), chitosan (e.g., highly purified cationic polysaccharide), pectin (or any carbohydrate that thickens like a gel or emulsifies when applied to nasal mucosa), a microsphere (e.g., starch, albumin, dextran, cyclodextrin), gelatin, a liposome, carbamer, polyvinyl alcohol, alginate, acacia, chitosans and/or cellulose (e.g., methyl or propyl; hydroxyl or carboxy; carboxymethyl or hydroxylpropyl), which are agents that enhance residence time in the nasal cavity. Asa further approach, increasing the viscosity of the formulation can also provide a means of prolonging contact of the agent with the nasal epithelium. The pharmaceutical composition can be formulated as a nasal emulsion, ointment or gel, which offer advantages for local application because of their viscosity.

Moist and highly vascularized membranes can facilitate rapid absorption; consequently, the pharmaceutical composition can optionally comprise a humectant, particularly in the case of a gel-based composition so as to assure adequate intranasal moisture content. Examples of suitable humectants include but are not limited to glycerin or glycerol, mineral oil, vegetable oil, membrane conditioners, soothing agents, and/or sugar alcohols (e.g., xylitol, sorbitol; and/or mannitol). The concentration of the humectant in the pharmaceutical composition will vary depending upon the agent selected and the formulation.

The pharmaceutical composition can also optionally include an absorption enhancer, such as an agent that inhibits enzyme activity, reduces mucous viscosity or elasticity, decreases mucociliary clearance effects, opens tight junctions, and/or solubilizes the active compound. Chemical enhancers are known in the art and include chelating agents (e.g., EDTA), fatty acids, bile acid salts, surfactants, and/or preservatives. Enhancers for penetration can be particularly useful when formulating compounds that exhibit poor membrane permeability, lack of lipophilicity, and/or are degraded by aminopeptidases. The concentration of the absorption enhancer in the pharmaceutical composition will vary depending upon the agent selected and the formulation.

To extend shelf life, preservatives can optionally be added to the pharmaceutical composition. Suitable preservatives include but are not limited to benzyl alcohol, parabens, thimerosal, chlorobutanol and benzalkonium chloride, and combinations of the foregoing. The concentration of the preservative will vary depending upon the preservative used, the compound being formulated, the formulation, and the like. In representative embodiments, the preservative is present in an amount of about 2% by weight or less.

The pharmaceutical composition can optionally contain an odorant, e.g., as described in EP 0 504 263 B1 to provide a sensation of odor, to aid in inhalation of the composition so as to promote delivery to the olfactory region and/or to trigger transport by the olfactory neurons.

As another option, the composition can comprise a flavoring agent, e.g., to enhance the taste and/or acceptability of the composition to the subject.

The invention also encompasses methods of intranasal administration of the pharmaceutical compositions of the invention. In representative embodiments, the compound is delivered by the intranasal route to the CNS, more specifically, the hypothalamus. According to exemplary methods, the pharmaceutical composition is administered to the upper third of the nasal cavity, to the superior meatus, the olfactory region and/or the sinus region of the nose. The olfactory region is a small area that is typically about 2-10 cm² in human (25 cm² in the cat) located in the upper third of the nasal cavity for deposition and absorption by the olfactory epithelium and subsequent transport by olfactory receptor neurons. Located on the roof of the nasal cavity, in the superior meatus, the olfactory region is desirable for delivery because it is the only known part of the body in which an extension of the CNS comes into contact with the environment (Bois et al., Fundamentals of Otolaryngology, p. 184, W.B. Saunders Co., Phila., 1989).

In particular embodiments, the pharmaceutical composition is administered to the subject in an effective amount, optionally, a treatment effective amount (each as described hereinabove).

Dosages of pharmaceutically active compositions can be determined by methods known in the art, see, e.g., Remington's Pharmaceutical Sciences (Maack Publishing Co., Easton, Pa.; 18^{th} edition, 1990).

A treatment effective amount will vary with the age and general condition of the subject, the severity of the condition being treated, the particular compound or composition being administered, the duration of the treatment, the nature of any concurrent treatment, the carrier used, and like factors within the knowledge and expertise of those skilled in the art. As appropriate, a treatment effective amount in any individual case can be determined by one of ordinary skill in the art by reference to the pertinent texts and literature and/or by using routine experimentation (see, e.g., Remington, The Science and Practice of Pharmacy (20^{th} ed. 2000)).

As a general proposition, a dosage from about 0.001, 0.01 or 0.1 to about 1, 2, 5, 10, 15, 20, 50, 75, 100, 200, 500 mg/kg body weight or more will be effective for treatment, with all weights being calculated based upon the weight of the active ingredient, including salts.

Exemplary dosages include from about 0.001, 0.01 or 0.1 to about 1, 5, 10 or 20 mg/dose, e.g., once, twice or three times daily, two to four times weekly, weekly, two to three times monthly or monthly.

Other suitable dosages are from about 0.001, 0.01 or 0.1 to about 1, 2, 3, 4, 5 or 10 mg/m² body surface area.

The compound can be administered for a sustained period, such as at least about one month, at least about 2 months, at least about 3 months, at least about 6 months, or at least about 12 months or longer (e.g., as a chronic life-long treatment).

Any suitable dosing schedule can be followed. For example, the dosing frequency can be a once weekly dosing. The dosing frequency can be a once daily dosing. The dosing frequency can be more than once weekly dosing. The dosing frequency can be more than once daily dosing, such as any one of 2, 3, 4, 5, or more than 5 daily doses. The dosing frequency can be 3 times a day. The dosing frequency can be three times a week dosing. The dosing frequency can be a four times a week dosing. The dosing frequency can be a two times a week dosing. The dosing frequency can be more than once weekly dosing but less than daily dosing. The dosing frequency can be a once monthly dosing. The dosing frequency can be a twice weekly dosing. The dosing frequency can be more than once monthly dosing but less than one weekly dosing. The dosing frequency can intermittent (e.g., one daily dosing for 7 days followed by no doses for 7 days, repeated for any 14 day time period, such as 2 months, 4 months, 6 months or more). The dosing frequency can be continuous (e.g., one weekly dosing for continuous weeks).

In other embodiments, the methods of the invention can be carried out on an as-needed basis by self-medication.

Any of the dosing frequencies described above can be used with any dosage amount described above. Further, any of the dosing frequencies and/or dosage amounts can be used with any of the compounds described herein.

The pharmaceutical composition can be delivered in any suitable volume of administration. In representative embodiments of the invention, the administration volume for intranasal delivery ranges from about 25 microliters to 200 microliters or from about 50 to 150 microliters in a laboratory animal such as a rat or mouse and from about 50, 100, 250 or 500 microliters to about 1, 2, 3, 3.5 or 4 milliliters in a human. Typically, the administration volume is selected to be large enough to allow for the dissolution of an effective amount of the active compound but sufficiently small to prevent therapeutically significant amounts of compound from escaping from the anterior chamber of the nose and/or draining into the throat, post nasally.

In particular embodiments, the compounds of the invention are delivered to the spinal cord or to the brain, more specifically, the brainstem (medulla oblongata and/or pons), midbrain (hypothalamus, thalamus, epithalamus, pituitary gland, substantia nigra and/or pineal gland), cerebellum, telencephalon (corpus striatum, cerebrum [including the occipital, temporal, parietal and/or frontal lobes], cortex, basal ganglia [including the striatum, which further includes the caudate nucleus and/or the putamen], hippocampus and/or amygdala), limbic system, neocortex, corpus striatum, cerebrum, and/or inferior colliculus. Delivery to the hypothalamus can optionally be to the mediobasal hypothalamus or ARC.

Any suitable method of intranasal delivery can be employed for delivery of the pharmaceutical compound. In particular embodiments, intranasal administration is by inhalation (e.g., using an inhaler, atomizer or nebulizer device), alternatively, by spray, tube, catheter, syringe, dropper, packtail, pledget, and the like. As a further illustration, the pharmaceutical composition can be administered intranasally as (1) nose drops, (2) powder or liquid sprays or aerosols, (3) liquids or semisolids by syringe, (4) liquids or semisolids by swab, pledget or other similar means of application, (5) a gel, cream or ointment, (6) an infusion, or (7) by injection, or by any means now known or later developed in the art. In particular embodiments, the method of delivery is by nasal drops, spray or aerosol. As used herein, aerosols can be used to deliver powders, liquids or dispersions (solids in liquid).

In representative embodiments, the pharmaceutical formulation is directed upward during administration, so as to enhance delivery to the upper third (e.g., the olfactory epithelium in the olfactory region) and the side walls (e.g., nasal epithelium) of the nasal cavity. Further, orienting the subject's head in a tipped-back position or orienting the subject's body in Mygind's position or the praying-to-Mecca position can be used to facilitate delivery to the olfactory region.

Many devices are known in the art for nasal delivery. Exemplary devices include particle dispersion devices, bidirectional devices, and devices that use chip-based ink-jet technologies. ViaNase (Kurve Technolgies, Inc., USA) uses controlled particle dispersion technology (e.g., an integrated nebulizer and particle dispersion chamber apparatus, for example, as described in International patent publication WO 2005/023335). Optinose and Optimist (OptiNose, AS, Norway) and DirectHaler (Direct-Haler AJS, Denmark) are examples of bidirectional nasal delivery devices. Ink-jet dispensers are described in U.S. Pat. No. 6,325,475 (MicroFab Technologies, Inc., USA) and use microdrops of drugs on a millimeter sized chip. Devices that rely on iontophoresis/phonophoresis/electrotransport are also known, as described in U.S. Pat. No. 6,410,046 (Intrabrain International NV, Curacao, AN). These devices comprise an electrode with an attached drug reservoir that is inserted into the nose. Iontophoresis, electrotransport or phonophoresis with or without chemical permeation enhancers can be used to deliver the drug to the target region (e.g., olfactory).

Nasal delivery devices are also described in U.S. Pat. No. 6,715,485 (OptiNose AS); U.S. Pat. No. 6,325,475 (Microfab Technologies, Inc.); U.S. Pat. No. 6,948,492 (University of Kentucky Research Foundation); U.S. Pat. No. 6,244,573 (LyteSyde, LLC); U.S. Pat. No. 6,234,459 (LyteSyde, LLC); U.S. Pat. No. 6,244,573 (LyteSyde, LLC); U.S. Pat. No. 6,113,078 (LyteSyde, LLC); U.S. Pat. No. 6,669,176 (LyteSyde, LLC); U.S. Pat. No. 5,724,965 (Respironics Inc.); and U.S. Patent Publications US2004/0112378 A1; US 2004/0112379 A1; US 2004/0149289 A1; US 2004/0112380 A1; US 2004;0182388 A1; US 2005/0028812 A1; US 2005/0235992 A1; US 2005/0072430 A1 and US 2005/0061324 A1.

As yet another aspect, the invention provides a method of operating an intranasal delivery device comprising a compound of the invention or pharmaceutically acceptable salt or prodrug thereof. In representative embodiments, the invention provides a method of operating an intranasal delivery device comprising a pharmaceutical composition formulated for intranasal delivery, the pharmaceutical composition comprising a compound of the invention or pharmaceutically acceptable-salt or prodrug thereof in a pharmaceutically acceptable carrier. Optionally, the device is configured and/or operated and/or the composition is formulated to enhance delivery to the upper third of the nasal cavity, superior meatus, olfactory region and/or the sinus region of the nose.

In particular embodiments, the invention provides a method of operating an intranasal delivery device, comprising: activating the intranasal delivery device to deliver a compound of the invention or pharmaceutically acceptable salt or prodrug thereof to a target location so that the compound or pharmaceutically acceptable salt or prodrug thereof is delivered to the CNS (e.g., the hypothalamus). Optionally, the compound is delivered as part of a pharmaceutical composition formulated for intranasal delivery. Optionally, the device is configured and/or operated and/or the composition is formulated to enhance delivery to the upper third of the nasal cavity, superior meatus, olfactory region and/or the sinus region of the nose along the olfactory neural pathway that has both intraneuronal and extraneuronal routes into the brain (Frey et al., (2002) Drug Delivery Tech. 2 (5): 46-49). According to this aspect of the invention, the activating step can further comprise positioning a unit dose container releasably holding the compound, pharmaceutically acceptable salt or prodrug thereof; nebulizing or atomizing the agent in the device; and releasing the nebulized or atomized agent intranasally.

### Pharmaceutical Formulations and Methods for Pulmonary Delivery.

The invention also encompasses pharmaceutical compositions formulated for pulmonary administration comprising one or more compounds that increase tricarboxylic acid (TCA) cycle flux through acetyl-CoA in the hypothalamus of a mammal in a pharmaceutically acceptable carrier. Compounds that can be used in the pharmaceutical compositions of the invention are discussed above. The one or more compounds can individually be prodrugs that are converted to the active compound in vivo. In particular embodiments, the invention provides a pharmaceutical composition formulated for pulmonary administration comprising a compound of the invention in a pharmaceutically acceptable excipient.

“Pulmonary administration” or “administration to the lungs,” and like terms, are used interchangeably herein. These terms refer to delivery of a composition to the lung(s) of a subject, e.g., the bronchi, bronchioli and/or alveoli. Generally, pharmaceutical compositions administered to the respiratory tract by oral or nasal inhalation travel through the upper airways (oropharynx and larynx), the lower airways which include the trachea followed by bifurcations into the bronchi and bronchioli and through the terminal bronchioli which in turn divide into respiratory bronchioli leading then to the ultimate respiratory zone, the alveoli or the deep lung. Absorption through the alveoli results from rapid dissolution of the formulation in the ultra-thin (0.1 μm) fluid layer of the alveolar lining of the lung. According to certain aspects of the invention, pulmonary administration is to the deep lung or alveoli. In particular embodiments of the invention, at least about 5%, 10%, 20%, 30%, 40%, 50% or more of the mass of particles deposits in the deep lung or alveoli. Deposition in the deep lung, for example the alveoli, can be influenced by a variety of factors including the delivery method, the characteristics of the delivery device (e.g., the size of the particles produced, the delivery velocity, and the like), and the characteristics of the delivered composition. Compositions and methods for achieving enhanced delivery to the deep lung or alveoli are discussed below.

In particular embodiments, the pharmaceutical composition is administered to the subject in an effective amount, optionally, a treatment effective amount. Dosages of pharmaceutically active compositions can be determined by methods known in the art, see, e.g., Remington's Pharmaceutical Sciences (Maack Publishing Co., Easton, Pa.; 18^{th} edition, 1990).

Aerosol dosage, formulations and delivery systems may be selected as described, for example, in Gonda, “Aerosols for delivery of therapeutic and diagnostic agents to the respiratory tract,” in Critical Reviews in Therapeutic Drug Carrier Systems, 6: 273-313, 1990; and in Moren, “Aerosol dosage forms and formulations,” in: Aerosols in Medicine. Principles, Diagnosis and Therapy, Moren, et al., Eds, Esevier, Amsterdam, 1985.

The pharmaceutical composition can be delivered in any suitable volume or mass (weight) of administration. In representative embodiments of the invention, the administration volume of liquid particles (e.g., liquid aerosol particles) in a single administration suitable for pulmonary delivery ranges from several microliters to several milliliters (e.g., from about 3 microliters to about 3, 4 or 5 milliliters). In other particular embodiments, the mass of solid particles (e.g., solid aerosol particles) in a single administration suitable for pulmonary delivery ranges from several micrograms to several milligrams (e.g., about 3 micrograms to about 3, 4 or 5 milligrams).

Pulmonary administration of any suitable formulation known or later discovered in the art is contemplated. For example, the composition can be formulated for nasal or oral administration to the lungs. In certain embodiments, the composition is formulated for oral inhalation. In other exemplary embodiments, the composition is administered as an aerosol solution, a suspension, or a dry powder of respirable particles containing the active agent, which the subject inhales. The respirable particles can be liquid or solid; optionally, the respirable particles are a dry powder or liquid aerosol. Further, in certain embodiments the respirable particles described above can be administered by oral inhalation.

For the purposes of the present invention, the term “aerosol” includes any gas-borne suspended phase, which is capable of being inhaled into the bronchioles or nasal passages. Specifically, an aerosol includes a gas-borne suspension of droplets, as can be produced in a metered dose inhaler, nebulizer, mist sprayer, or the like. The term “aerosol” also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn, et al. (1992) J. Pharmacol. Toxicol. Methods 27:143-159.

In particular embodiments, the pulmonary formulation comprises a dispersible dry powder. “Dispersibility” or “dispersible” or equivalent terms means a dry powder having a moisture content of less than about 10% by weight (% w) water, usually below about 5% w, or less than about 3% w; a particle size of about 1-5 μm mass median diameter (MMD), 1-4 μm MMD or 1-3 μm MMD; a delivered dose of about >5%, >10%, >15%, >20%, >30%, >40% or >50%; and an aerosol particle size distribution of about 1-5 μm mass median aerodynamic diameter (MMAD), 1.5-4.5 μm MMAD, or 1.5-3 μm MMAD. Methods and compositions for improving dispersibility are disclosed in U.S. Pat. Nos. 6,136,346; 6,358,530 and 6,582,729; the disclosures of which are hereby incorporated by reference.

The term “powder” as used herein means a composition that consists of finely dispersed solid particles that are free flowing and capable of being readily dispersed in an inhalation device and subsequently inhaled by a subject so that the particles reach the lungs, and optionally permit penetration into the alveoli. In particular embodiments, the average particle size is less than about 10, 7.5, or 5 μm in diameter with a relatively uniform spheroidal shape distribution. Generally, the particle size distribution is between about 0.1 μm and about 5 μm, particularly about 0.3 μm to about 5 μm or about 1 μm to about 3 μm.

The term “dry” means that the composition has a moisture content such that the particles are readily dispersible in an inhalation device to form an aerosol. This moisture content is generally below about 10% by weight (% w) water, usually below about 5% w or below about 3% w.

In the dry state, the powder may be in crystalline or amorphous form.

A treatment effective amount of active compound will vary in the composition depending on the biological activity of the compound(s) employed and the amount needed in a unit dosage form. Because the composition is dispersible, it is generally advantageous that it is manufactured in a unit dosage form in a manner that allows for ready manipulation by the formulator and by the consumer. A unit dosage will typically be between about 0.5 mg and 15 mg, more particularly between about 2 mg and 10 mg, of total material in the dry powder composition.

Aerosols of liquid particles containing the active agent can be produced by any suitable aerosolization means, such as with a pressure-driven aerosol nebulizer, an electrostatic nebulizer, an ultrasonic nebulizer, a pressured/volatile gas-filled metered dose inhaler (MDI), a piston-driven system with a grid or laser-drilled holes, or devices that rely upon the subject's inspiratory flow, as are known to those of skill in the art. See, e.g., U.S. Pat. No. 4,501,729.

Aerosols of solid particles containing the active agent can likewise be produced with any solid particulate medicament aerosol generator, by techniques known in the pharmaceutical art. See, for example, U.S. Pat. No. 6,169,068, U.S. Pat. No. 6,334,999 and U.S. Pat. No. 6,797,258; the disclosure of which is incorporated herein by reference.

As further examples, solid particles can be delivered from an inhalation device such as a dry powder inhaler (DPI) or a MDI.

Other suitable inhalers are described in U.S. Pat. No. 4,069,819, U.S. Pat. No. 4,995,385, and U.S. Pat. No. 5,997,848. Further examples include, but are not limited to, the Spinhaler®. (Fisons, Loughborough, U.K.), Rotahaler®. (Glaxo-Wellcome, Research Triangle Technology Park, NC), FlowCaps®. (Hovione, Loures, Portugal), Inhalator® (Boehringer-Ingelheim, Germany), and the Aerolizer® (Novartis, Switzerland), the diskhaler (Glaxo-Wellcome, RTP, NC) and others, such as known to those skilled in the art.

Dry powder dispersion devices for medicaments are described in a number of patent documents. U.S. Pat. No. 3,921,637 describes a manual pump with needles for piercing through a single capsule of powdered medicine. The use of multiple receptacle disks or strips of medication is described in. European Patent Application No. EP 0 467 172 (where a reciprocatable punch is used to open a blister pack); International Patent Publication Nos. WO 91/02558 and WO 93/09832; U.S. Pat. Nos. 4,627,432; 4,811,731; 5,035,237; 5,048,514; 4,446,862; 5,048,514; and 4,446,862. Other patents that show puncturing of single medication capsules include U.S. Pat. Nos. 4,338,931; 3,991,761; 4,249,526; 4,069,819; 4,995,385; 4,889,114; and 4,884,565; and European Patent Application No. EP 469 814. International Patent Publication No. WO 90/07351 describes a hand-held pump device with a loose powder reservoir.

A dry powder sonic velocity disperser is described in Witham and Gates, Dry Dispersion with Sonic Velocity Nozzles, presented at the workshop on Dissemination Techniques for Smoke and Obscurants, Chemical Systems Laboratory, Aberdeen Proving Ground, Maryland, Mar. 14-16, 1983.

U.S. Pat. Nos. 4,926,852 and 4,790,305 describe a type of “spacer” for use with a metered dose inhaler. The spacer defines a large cylindrical volume which receives an axially directed burst of drug from a propellant-driven drug supply. U.S. Pat. No. 5,027,806 is an improvement over the '852 and '305 patents, having a conical holding chamber that receives an axial burst of drug. U.S. Pat. No. 4,624,251 describes a nebulizer connected to a mixing chamber to permit a continuous recycling of gas through the nebulizer. European Patent Application No. 0 347 779 describes an expandable spacer for a metered dose inhaler having a one-way valve on the mouthpiece. International Patent Publication No. WO 90/07351 describes a dry powder oral inhaler having a pressurized gas source (a piston pump) which draws a measured amount of powder into a venturi arrangement.

Stribling et al. (1992) J. Biopharm. Sci. 3:255-263, describes the aerosol delivery of plasmids carrying a chloramphenicol acetyltransferase (CAT) reporter gene to mice. The plasmids were incorporated in DOTMA (N-[1-(2-, 3-dioleyloxy)propyl]-N,N,N-trimethylammonium chloride) or cholesterol liposomes, and aqueous suspensions of the liposomes were nebulized into a small animal aerosol delivery chamber. Mice breathing the aerosol were found to at least transiently express CAT activity in their lung cells. Rosenfeld et al. (1991) Science: 252:431-434, describes the in vivo delivery of an alpha-1 antitrypsin gene to rats, with secretion of the gene product being observable for at least one week. The gene was diluted in saline and instilled directly into the rat trachea.

Patton and Platz (1992) Adv. Drug Deliver. Rev. 8:179-196, describe methods for delivering proteins and polypeptides by inhalation to the deep lung.

The aerosolization of protein therapeutic agents, including alpha-1 antitrypsin, is disclosed in European Patent Application No. EP 0 289 336. In general, nebulizers can be advantageous for delivery of liquid compositions comprising polypeptides, as nebulizers are gentler on the pharmaceutical composition than, for example, a MDI device.

There are several considerations that bear upon the design and operation of the delivery device. To illustrate, unlike dry powder administration, for liquids it is the device that determines particle size, usually as a function of the diameter of the delivery port, mesh or grid. Liquid particles (i.e., droplets) having a desired size as described herein can be achieved by selection of a suitable delivery device based on considerations well-known in the art.

Delivery velocity is another factor to consider for pulmonary delivery. Even particles of an optimum size will rebound from the soft palate, and therefore not travel down the trachea, if they are delivered at too high a velocity. On the other hand, particles that are delivered too slowly will not enter the respiratory tract at all (e.g., in the case of oral inhalation, they will land on the tongue).

Delivery devices and methods can also be selected to time delivery of the pharmaceutical composition with the breathing cycle. Some devices incorporate firmware that measures the timing of the patient's breathing cycle and optimizes pulsed drug delivery to achieve efficient delivery. More commonly, devices (included pulsed nebulizers, MDIs and dry powder devices) rely on a learned coordination by the patient of drug delivery with inspiration. MDIs are available that provide a mixing cylinder placed between the delivery device and the mouthpiece to improve dispersion of the aerosol and reduce the need to time delivery with the breathing cycle.

As yet another approach, some devices incorporate a heating device to warm the pharmaceutical composition to body temperature during delivery, which results in more efficient pulmonary delivery.

The compositions of the invention can be formulated for pulmonary administration in a pharmaceutical carrier in accordance with known techniques. See, e.g., Remington, The Science And Practice of Pharmacy (20^{th} edition, 2000). Suitable nontoxic pharmaceutically acceptable carriers for pulmonary administration will be apparent to those skilled in the art of pulmonary pharmaceutical formulations (see, e.g., Remington's Pharmaceutical Sciences (Maack Publishing Co., Easton latest edition).

The composition may optionally be combined with pharmaceutical carriers or excipients that are suitable for pulmonary administration. Such carriers may serve simply as bulking agents when it is desired to reduce the pharmaceutical concentration in the powder which is being delivered to a patient, but may also serve to enhance the stability of the compositions and to improve the dispersibility of the powder within a powder dispersion device in order to provide more efficient and reproducible delivery of the powder and to improve handling characteristics such as flowability and consistency to facilitate manufacturing and powder filling.

Such carrier materials may be combined with the drug prior to spray drying, e.g., by adding the carrier material to the purified bulk solution. In that way, the carrier particles will be formed simultaneously with the drug particles to produce a homogeneous powder. Alternatively, the carriers may be separately prepared in a dry powder form and combined with the dry powder drug by blending. The powder carriers will usually be crystalline (to avoid water absorption), but might in some cases be amorphous or mixtures of crystalline and amorphous. The size of the carrier particles may be selected to improve the flowability of the drug powder, typically being in the range from about 25 μm to 100 μm. One suitable carrier material is crystalline lactose having a size in the above-stated range.

The active compound(s) can be present in the formulation in any suitable amount, for example, a range from about 0.05, 0.1, 0.5 or 1% to about 50, 60, 70, 80, 90, 95, 97 or 99% (w/w or w/v).

The compound can have any suitable molecular weight. According to certain embodiments of the invention, the compound has a molecular weight of less than about 10 kiloDalton (kD), 7.5 kD, 5 kD, 2 kD, 1 kD, 500 Daltons or less.

The pharmaceutical composition can further have any suitable osmolarity, for example, in the range of about 100 to 600 mOsM, about 150 to 450 mOsM, or about 175 to 310 mOsM.

The formulation can further comprise one or more component(s) that promote(s) the fast release of the active compound(s) into the blood stream. In one embodiment, a therapeutic plasma concentration is achieved in less than about 10 minutes, 5 minutes, 2 minutes or even sooner after administration.

In particular embodiments, the formulation includes one or more phospholipids, such as, for example, a phosphatidylcholine, phosphatidylethanolamine, phosphatidylglycerol, phosphatidylserine, phosphatidylinositol or a combination thereof. For example, the phospholipids can be endogenous to the lung. Combinations of phospholipids can also be employed. Specific examples of phospholipids are shown in Table 1.

TABLE 1

| | |
|---|---|
| Dilaurylolyphosphatidylcholine (C12; 0) | DLPC |
| Dimyristoylphosphatidylcholine (C14; 0) | DMPC |
| Dipalmitoylphosphatidylcholine (C16:0) | DPPC |
| Distearoylphosphatidylcholine (18:0) | DSPC |
| Dioleoylphosphatidylcholine (C18:1) | DOPC |
| Dilaurylolylphosphatidylglycerol | DLPG |
| Dimyristoylphosphatidylglycerol | DMPG |
| Dipalmitoylphosphatidylglycerol | DPPG |
| Distearoylphosphatidylglycerol | DSPG |
| Dioleoylphosphatidylglycerol | DOPG |
| Dimyristoyl phosphatidic acid | DMPA |
| Dipalmitoyl phosphatidic acid | DPPA |
| Dimyristoyl phosphatidylethanolamine | DMPE |
| Dipalmitoyl phosphatidylethanolamine | DPPE |
| Dimyristoyl phosphatidylserine | DMPS |
| Dipalmitoyl phosphatidylserine | DPPS |
| Dipalmitoyl sphingomyelin | DPSP |
| Distearoyl sphingomyelin | DSSP |

The phospholipid can be present in the formulation in any suitable amount, e.g., an amount ranging from about 0, 1, 5 or 10% to about 50, 60, 70, 80 or 90% (w/w or w/v).

The phospholipids or combinations thereof can be selected to impart rapid or controlled-release properties to the formulation. Particles having controlled-release properties and methods of modulating release of a biologically active agent are described in U.S. patent application Ser. No. 09/644,736 and U.S. patent Publication No. 20010036481; the disclosures of which are incorporated herein by reference. Rapid release can be obtained, for example, by including in the formulation phospholipids characterized by low transition temperatures.

Rapid release can also be achieved by administering formulations comprising nanoparticles (e.g., because of large surface area) and formulations in the form of solutions.

Nanoparticles, microspheres, cyclodextrins and liposomes can be used as vehicles for controlled-release delivery.

In another embodiment, rapid and controlled-release of the active compound(s) are coupled in a single course of therapy.

The formulation can further include a surfactant. As used herein, the term “surfactant” refers to any agent that preferentially absorbs to an interface between two immiscible phases, such as the interface between water and an organic polymer solution, a water/air interface or organic solvent/air interface. Surfactants generally possess a hydrophilic moiety and a lipophilic moiety, such that, upon absorbing to microparticles, they tend to present moieties to the external environment that do not attract similarly-coated particles, thus reducing particle agglomeration. Surfactants may also promote absorption of a therapeutic or diagnostic agent and increase bioavailability of the agent.

In addition to lung surfactants, such as, for example, the phospholipids discussed above, suitable surfactants include but are not limited to hexadecanol; fatty alcohols such as polyethylene glycol (PEG) and acetyl alcohol; polyoxyethylene-9-lauryl ether; a surface active fatty acid, such as palmitic acid or oleic acid; glycocholate; surfactin; a poloxomer; a sorbitan fatty acid ester such as sorbitan trioleate (Span 85); and tyloxapol.

The surfactant can be present in the formulation in any suitable amount, for example, an amount ranging from about 0, 1, 5 or 10% to about 50, 60, 70, 80 or 90% (w/w or w/v).

Methods of preparing and administering particles including surfactants, in particular phospholipids, are disclosed in U.S. Pat. No. 5,855,913 and U.S. Pat. No. 5,985,309; the disclosures of both are incorporated herein by reference in their entireties.

Additives can be included for conformational stability during spray drying and for improving dispersibility of powders. One such group of additives includes amino acid(s), in particular, hydrophobic amino acid(s). Suitable amino acids include naturally occurring and non-naturally occurring amino acids. Specific examples of amino acids which can be employed include, but are not limited to: alanine, glycine, proline, cysteine, methionine, valine, leucine, tyrosine, isoleucine, phenylalanine, and tryptophan. Non-naturally occurring amino acids include, for example, β-amino acids. Both D, L and racemic configurations of amino acids can be employed. Suitable amino acids can also include amino acid analogs as described above.

Hydrophobicity is generally defined with respect to the partition of an amino acid between a nonpolar solvent and water. Hydrophobic amino acids are those amino acids that show a preference for the nonpolar solvent. Relative hydrophobicity of amino acids can be expressed on a hydrophobicity scale on which glycine has the value 0.5. On such a scale, amino acids that have a preference for water have values below 0.5 and those that have a preference for nonpolar solvents have a value above 0.5. As used herein, the term “hydrophobic amino acid” refers to an amino acid that on the hydrophobicity scale has a value greater or equal to 0.5, in other words, has a tendency to partition in the nonpolar solvent that is at least equal to that of glycine.

In representative embodiments, combinations of hydrophobic amino acids are employed. Furthermore, in other embodiments, combinations of hydrophobic and hydrophilic (preferentially partitioning in water) amino acids, where the overall combination is hydrophobic, are employed.

The amino acid can be present in the pulmonary formulations in any suitable amount, for example, an amount of at least about 10% (w/w or w/v). In particular embodiments, the amino acid is present in the formulation in an amount ranging from about 20% to about 80% (w/w or w/v). The salt of a hydrophobic amino acid can be present in the formulation in any suitable amount, for example, an amount of at least about 10% (w/w or w/v). In illustrative embodiments, the amino acid salt is present in the formulation in an amount ranging from about 20% to about 80% (w/w or w/v). Methods of forming and delivering particles that include an amino acid are described in U.S. Pat. No. 6,586,008 and U.S. patent application Ser. No. 09/644,320; the teachings of which are incorporated herein by reference.

In another embodiment of the invention, the formulation includes a carboxylate moiety and/or a multivalent metal salt. Such compositions are described, for example, in U.S. Pat. No. 6,749,835; the disclosure of which is incorporated herein by reference. In one particular embodiment, the formulation includes sodium citrate and/or calcium chloride.

The pulmonary formulation can further comprise carriers including but not limited to stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-β-cyclodextrin; and polysaccharides, such as raffinose, maltodextrins, dextrans, and the like; alditols, such as mannitol, xylitol, and the like. Suitable polypeptides include aspartame. Amino acids include alanine and glycine.

The pharmaceutical composition can have any suitable pH. In representative embodiments, the pH of the pharmaceutical composition ranges from about 4, 4.5, 5 or 5.5 to about 6, 6.5, 7, 7.5 or 8. Exemplary pH ranges include without limitation from about pH 4.5 to about pH 8 and from about pH 5.5 to about pH 7.5. Those skilled in the art will appreciate that because the volume of the pharmaceutical composition administered is generally small (e.g., less than about 5 milliliters), secretions from the respiratory tract may alter the pH of the administered dose. Such alterations can affect the concentration of un-ionized drug available for absorption. Accordingly, in representative embodiments, the pharmaceutical composition further comprises a buffer to maintain or regulate pH in situ. Typical buffers include but are not limited to organic salts, e.g., prepared from organic acids and bases, such as acetate, citrate, prolamine, carbonate, ascorbate and phosphate buffers.

Other materials, including materials that promote fast release kinetics of the active compound can also be employed. For example, biocompatible, and optionally biodegradable polymers can be employed. Illustrative formulations including such polymeric materials are described in U.S. Pat. No. 5,874,064; the disclosure of which is incorporated herein by reference in its entirety.

The formulation can further include a material such as, for example, dextran, polysaccharides, lactose, trehalose, cyclodextrins, proteins, peptides, polypeptides, fatty acids, inorganic compounds and/or phosphates.

To extend shelf life, preservatives can optionally be added to the pharmaceutical composition. Suitable preservatives include but are not limited to benzyl alcohol, parabens, thimerosal, chlorobutanol and benzalkonium chloride, and combinations of the foregoing. The concentration of the preservative will vary depending upon the preservative used, the compound being formulated, the formulation, and the like. In representative embodiments, the preservative is present in an amount of 2% by weight or less. In certain embodiments, the pharmaceutical composition is sufficiently stable as to not require the addition of preservatives. The absence of preservatives can be advantageous since preservatives may raise safety and toxicity issues, especially to the lung.

In particular embodiments, the formulation is substantially free of any penetration enhancers. The use of penetration enhancers in formulations for the lungs is often undesirable because the epithelial cell layer in the lung can be adversely affected by such surface active compounds.

As another option, the composition can comprise a flavoring agent, e.g., to enhance the taste and/or acceptability of the composition to the subject.

In the case of active, compounds comprising polypeptides, it is generally desirable that the compound be shielded from leukocyte proteases in the lung and/or have a structure that is resistant to proteolytic degradation. To illustrate; the compound can be protected from proteolytic cleavage by encapsulation, for example, in lysosomes. As another option, the compound can be formulated with a protease inhibitor, such as benzamidine or a derivative thereof (see, e.g., Pauls et al., (2004) Front. Med. Chem. 1:129-152). As still another approach, polypeptides can be synthesized with modified peptide bonds and/or with blocked or otherwise modified amino and/or carboxyl termini that are resistant to proteolytic cleavage.

In representative embodiments, the active compound is lipophilic to promote absorption. In general, nonpolar compounds more readily cross the mucosal lining and the epithelial cell layer in the lungs. In other embodiments, uptake of non-lipophilic compounds is enhanced by combination with a lipophilic substance. Lipophilic substances that can enhance absorption of the compound include but are not limited to fatty acids (e.g., palmitic acid), gangliosides (e.g., GM-1), phospholipids (e.g., phosphatidylserine), and emulsifiers (e.g., polysorbate 80), bile salts such as sodium deoxycholate, and detergent-like substances including, for example, polysorbate 80 such as Tween™, octoxynol such as Triton™ X-100, and sodium tauro-24,25-dihydrofusidate (STDHF). See Lee et al., Biopharm., April 1988 issue:3037.

In particular embodiments of the invention, the active compound is combined with micelles comprised of lipophilic substances, e.g., to achieve a uniform emulsion. Such micelles can modify the permeability of the alveoli membrane to enhance absorption of the compound. Suitable lipophilic micelles include without limitation gangliosides (e.g., GM-1 ganglioside), and phospholipids (e.g., phosphatidylserine). Bile salts and their derivatives and detergent-like substances can also be included in the micelle formulation. The active compound can be combined with one or several types of micelles, and can further be contained within the micelles or associated with their surface.

Alternatively, the active compound can be combined with liposomes (lipid vesicles) to enhance absorption. The active compound can be contained or dissolved within the liposome and/or associated with its surface. Suitable liposomes include phospholipids (e.g., phosphatidylserine) and/or gangliosides (e.g., GM-1). For methods of making phospholipid vesicles, see for example, U.S. Pat. No. 4,921,706 to Roberts et al., and U.S. Pat. No. 4,895,452 to Yiournas et al. Bile salts and their derivatives and detergent-like substances can also be included in the liposome formulation.

The pharmaceutical composition can be selected to enhance delivery to the desired target regions, e.g., the deep lung or alveoli. In particular embodiments, the liquid or dry powder particles, optionally liquid or dry powder aerosol particles, have a tap density less than about 0.4, 0.2 or even 0.1 g/cm³. Particles that have a tap density of less than about 0.4 g/cm³ are referred to herein as “aerodynamically light particles”. Tap density can be measured by using instruments known to those skilled in the art such as but not limited to the Dual Platform Microprocessor Controlled Tap Density Tester (Vankel, N C) or a GeoPyc™ instrument (Micrometrics Instrument Corp., Norcross, Ga.). Tap density is a standard measure of the envelope mass density. Tap density can be determined using the method of USP Bulk Density and Tapped Density, United States Pharmacopeia convention, Rockville, Md., 10^{th} Supplement, 4950-4951, 1999. Features that can contribute to low tap density include irregular surface texture and porous structure.

The envelope mass density of an isotropic particle is defined as the mass of the particle divided by the minimum sphere envelope volume within which it can be enclosed. In one embodiment of the invention, the particles have an envelope mass density of less than about 0.4 g/cm³.

In particular embodiments, aerodynamically light particles have a size, e.g., a volume median geometric diameter (VMGD), of at least about 5 μm. In one embodiment, the VMGD is from about 5 μm to about 30 μm. In another embodiment of the invention, the particles have a VMGD ranging from about 10 μm to about 30 μm. In other embodiments, the particles have a median diameter, mass median diameter (MMD), a mass median envelope diameter (MMED) or a mass median geometric diameter (MMGD) of at least 5 μm, for example from about 5 μm to about 30 μm.

The diameter of spray-dried particles, for example, the VMGD, can be measured using an electrical zone sensing instrument such as a Multisizer IIe, (Coulter Electronic, Luton, Beds, England), or a laser diffraction instrument (for example Helos, manufactured by Sympatec, Princeton, N.J.). Other instruments for measuring particle diameter are well known in the art. The diameter of particles in a sample will range depending upon factors such as particle composition and methods of synthesis. The distribution of size of particles in a sample can be selected to permit optimal deposition to targeted sites within the lungs.

In representative embodiments, aerodynamically light liquid or dry powder particles have a “mass median aerodynamic diameter” (MMAD), also referred to herein as “aerodynamic diameter”, between about 1 μm and about 5 μm. In another embodiment of the invention, the MMAD is between about 1 μm and about 3 μm. In a further embodiment, the MMAD is between about 3 μm and about 5 μm.

Experimentally, aerodynamic diameter can be determined by employing a gravitational settling method, whereby the time for an ensemble of particles to settle a certain distance is used to infer directly the aerodynamic diameter of the particles. An indirect method for measuring the mass median aerodynamic diameter (MMAD) is the multi-stage liquid impinger (MSLI).

The aerodynamic diameter, dₐₑᵣ, can be calculated from the equation:

dₐₑᵣ=d_{g}√ρₜₐₚ

where d_{g} is the geometric diameter, for example the MMGD, and ρ is the powder density.

Particles that have a tap density less than about 0.4 g/cm³, a median geometric diameter of at least about 5 μm, and/or an MMAD of between about 1 μm and about 3 or 5 μm, are more likely of escaping inertial and gravitational deposition in the oropharyngeal region, and are targeted to the airways, particularly the deep lung. In particular embodiments, the use of larger, more porous particles can be advantageous since they are generally able to aerosolize more efficiently than smaller, denser aerosol particles. In certain embodiments of the invention, such larger more porous particles are used as a vehicle for the delivery of dry powders.

In other representative embodiments, larger particles which are less porous, but which are effectively microspheres containing a suspension of dry particles or droplets, may also possess a density less than about 0.4 g/cm³, or preferably less than about 0.15 g/cm³. Such particles are most efficiently delivered to the deep lung if they possess a geometric diameter from about 4 μm to greater than about 8 μm.

In embodiments of the invention, the particles have an MMAD of about 1 to 5 μm, more particularly about 1 to 3 μm. The particles can be liquid or dry powder.

In embodiments in which the particles to be delivered are composed of an aerosol generated from a liquid (e.g., from an aqueous solution), the particles generally have a density greater than 1 g/cm³ and less than about 1.2 g/cm³. In these embodiments, the particles typically have a geometric diameter ranging from about 1 μm to about 5 μm or from about 1 μm to about 3 μm for delivery to the deep lung or alveoli.

In other embodiments, the particles generally have a median diameter of at least about 5 μm, and more particularly about 15-20 μm, and are generally more likely to avoid phagocytic engulfment by alveolar macrophages and clearance from the lungs, due to size exclusion of the particles from the phagocytes cytosolic space. According to this embodiment, the particles generally have a low density, e.g., dry powder particles or a suspension of microspheres.

The particles can be fabricated with the appropriate material, surface roughness, diameter and tap density for localized delivery to selected regions of the lungs such as the deep lung or upper or central airways. For example, higher density or larger particles may be used for upper airway delivery, or a mixture of varying sized particles in a sample, provided with the same or different therapeutic agent may be administered to target different regions of the lung in one administration. Particles having an MMAD ranging from about 3 to about 5 μm are generally suitable for delivery to the central and upper airways. Particles having an MMAD ranging from about 1 to about 3 μm or about 5 μm are generally suitable for delivery to the deep lung. Inertial impaction and gravitational settling of aerosols are predominant deposition mechanisms in the airways and acini of the lungs during normal breathing conditions. Edwards, D. A., J Aerosol Sci., 26: 293-317 (1995). The importance of both deposition mechanisms increases in proportion to the mass of aerosols and not to particle (or envelope) volume. Since the site of aerosol deposition in the lungs is influenced by the mass of the aerosol (at least for particles of mean aerodynamic diameter greater than approximately 1 μm), diminishing the tap density by increasing particle surface irregularities and particle porosity permits the delivery of larger particle envelope volumes into the lungs, all other physical parameters being equal.

The low tap density particles have a small aerodynamic diameter in comparison to the actual envelope sphere diameter. The aerodynamic diameter, dₐₑᵣ, is related to the envelope sphere diameter, d (Gonda, I., “Physico-chemical principles in aerosol delivery,” in Topics in Pharmaceutical Sciences 1991 (eds. D. J. A. Crommelin and K. K. Midha), pp. 95-117, Stuttgart: Medpharm Scientific Publishers, 1992)), by the formula:

dₐₑᵣ=d√ρ.

where the envelope mass ρ is in units of g/cm³. Maximal deposition of monodispersed aerosol particles in the alveolar region of the human lung (˜60%) occurs for an aerodynamic diameter of approximately dₐₑᵣ=3 μm. Heyder, J. et al., J Aerosol Sci., 17: 811-825 (1986). Due to their small envelope mass density, the actual diameter d of aerodynamically light particles comprising a monodisperse inhaled powder that will exhibit maximum deep-lung deposition is:

d=3/√ρμm (where ρ<1 g/cm³);

where d is greater than 3 μm. For example, aerodynamically light particles that display an envelope mass density, ρ=0.1 g/cm³, will exhibit a maximum deposition for particles having envelope diameters as large as 9.5 μm. The increased particle size diminishes interparticle adhesion forces. Visser, J., Powder Technology, 58: 1-10. Thus, large particle size generally increases efficiency of aerosolization to the deep lung for particles of low envelope mass density, in addition to contributing to lower phagocytic losses.

The aerodynamic diameter can be calculated to provide for maximum deposition within the lungs. Previously this was achieved by the use of very small particles of less than about five microns in diameter, preferably between about one and about three microns, which particles may then be subject to phagocytosis. For the delivery of formulations composed of microspheres or particles formulated for the delivery of a dry powder, particles that have a larger diameter, but that are sufficiently light (hence the characterization “aerodynamically light”), can result in an equivalent delivery to the lungs, with a lower susceptibility to phagocystosis. Improved delivery can be obtained by using particles with a rough or uneven surface, which also have a lower susceptibility for phagocystosis. In another embodiment of the invention, the particles have an envelope mass density, also referred to herein as “mass density” of less than about 0.4 g/cm³. In particular embodiments, particles have a mean diameter of between about 5 μm and about 30 μm. Mass density and the relationship between mass density, mean diameter and aerodynamic diameter are discussed in U.S. application Ser. No. 08/655,570, which is incorporated herein by reference. In a representative embodiment, the particles have a mass density less than about 0.4 g/cm³, a mean geometric diameter of between about 5 μm and about 30 μm and MMAD between about 1 μm and about 5 μm.

Suitable particles can be fabricated or separated, for example by filtration or centrifugation, to provide a particle sample with a preselected size distribution. For example, greater than about 30%, 50%, 70%, 80%, 90% or 95% of the particles in a sample can have a diameter within a selected range of at least about 5 μm. The selected range within which a certain percentage of the particles fall may be for example, between about 5 and about 30 μm, or between about 5 and about 15 μm. In one embodiment, at least a portion of the particles have a diameter between about 9 and about 11 μm. Optionally, the particle sample also can be fabricated wherein at least about 75%, 85%, 90%, or optionally about 95% or about 99%, have a diameter within the selected range. The presence of the higher proportion of the aerodynamically light, larger diameter particles in the particle sample can enhance the delivery of therapeutic or diagnostic agents incorporated therein to the deep lung. Large diameter particles generally mean particles having a median geometric diameter of at least about 5 μm.

Properties of the particles facilitate delivery to subjects with highly compromised lungs where other particles prove ineffective for those lacking the capacity to strongly inhale, such as young patients, old subjects, infirm subjects, or subjects with asthma or other breathing difficulties. Further, subjects suffering from a combination of ailments may simply lack the ability to sufficiently inhale. Thus, using the methods and particles described above, even a weak inhalation is sufficient to deliver the desired dose.

Alternatively, in other embodiments, smaller high-density particles that have sufficient momentum to achieve deep lung or alveoli delivery can be used.

Particles can be prepared by any method known in the art. In representative embodiments, suitable particles are fabricated by spray drying. The spray drying can be done under conditions that result in a substantially amorphous powder of homogeneous constitution having a particle size that is respirable, a low moisture content and flow characteristics that allow for ready aerosolization.

In one embodiment, the method includes forming a mixture including one or more compounds of the invention and a surfactant, such as, for example, the surfactants described above. The mixture employed in spray drying can include an organic or aqueous-organic solvent.

Suitable organic solvents that can be employed include but are not limited to alcohols for example, ethanol, methanol, propanol, isopropanol, butanols, and others. Other organic solvents include but are not limited to perfluorocarbons, dichloromethane, chloroform, ether, ethyl acetate, methyl tert-butyl ether and others. Co-solvents include an aqueous solvent and an organic solvent, such as, but not limited to, the organic solvents as described above. Aqueous solvents include water and buffered solutions. In one embodiment, an ethanol water solvent is preferred with the ethanol:water ratio ranging from about 50:50 to about 90:10 ethanol:water.

The spray drying mixture can have a neutral, acidic or alkaline pH (e.g., from about pH 3 to about pH 10). Optionally, a pH buffer can be added to the solvent or co-solvent or to the formed mixture.

Suitable spray-drying techniques are described, for example, by K. Masters in “Spray Drying Handbook”, John Wiley & Sons, New York, 1984. Generally, during spray-drying, heat from a hot gas such as heated air or nitrogen is used to evaporate the solvent from droplets formed by atomizing a continuous liquid feed. Other spray-drying techniques are well known to those skilled in the art. In a preferred embodiment, a rotary atomizer is employed. An example of suitable spray driers using rotary atomization includes the Mobile Minor spray drier, manufactured by Niro, Denmark. The hot gas can be, for example, air, nitrogen or argon.

The particles can be fabricated with a rough surface texture to reduce particle agglomeration and improve flowability of the powder. The spray-dried particles can have improved aerosolization properties. The spray-dried particle can be fabricated with features which enhance aerosolization via dry powder inhaler devices, and lead to lower deposition in the mouth, throat and inhaler device.

Alternatively, dry powder compositions may be prepared by other processes such as lyophilization and jet milling as disclosed in International Patent Publication No. WO 91/16038, the disclosure of which is hereby incorporated by reference.

In other embodiments of the invention, the formulation is administered as a liquid, an emulsion, or a dispersion. Liquid-born agents can be delivered to the lungs by any method known in the art, e.g., by recirculation in and out of the lungs (e.g., by liquid lavage or liquid ventilation) or maintained in a static system (i.e., non-recirculated) for extended periods of time. For example, in representative embodiments, a liquid can be instilled via a lavage tube. As another option, a liquid aerosol can be instilled via a respirator.

U.S. Pat. No. 6,242,472 describes the delivery of therapeutic agents in a liquid carrier such as saline, silicone, vegetable oil or perfluorochemicals (e.g., perfluorocarbon), e.g., in the form of an emulsion or a dispersion, for delivery to the pulmonary air passages; the disclosure of this patent is incorporated by reference herein in its entirety.

The active compound can be present in the liquid in any suitable form, e.g., bulk form, a suspension, a dispersion, a liquid form, an emulsion, and/or an encapsulized form. Moreover, the selected compound(s) can be incorporated into the liquid medium by any suitable technique. Examples of suitable incorporation techniques include, but are not limited to, injection, blending, or dissolving.

Liquids can be selectively directed to specific regions of the subject's lungs by a number of conventional means, such as a bronchoscope or a catheter.

The methods of delivery to the lungs can be carried out once or multiple times, and can further be carried out daily, every other day, etc., with a single administration or multiple administrations per day of administration, (e.g., 2, 3, 4 or more times per day of administration). In representative embodiments, the methods of the invention can be carried out on an as-needed basis by self-medication.

In particular embodiments, the methods of the invention comprise administering to the pulmonary system a therapeutic dose in a small number of breath-activated steps (e.g., less than 5, 4, or 3), and even in one or two breath-activated step(s).

Particular methods include administering particles from a receptacle having, holding, containing, storing or enclosing a mass of particles, to a subject's lungs. In one example, at least 50% of the mass of the particles stored in the inhaler receptacle is delivered to a subject's lungs in a single, breath-activated step. In another embodiment, at least 10 milligrams of the active compound(s) is delivered by administering, in a single breath, to a subject's lungs particles enclosed in the receptacle. Amounts as high as 15, 20, 25, 30, 35, 40 and 50 milligrams or more can be delivered.

In one embodiment, delivery to the pulmonary system of particles in a single, breath-actuated step is enhanced by employing particles that are dispersed at relatively low energies, such as, for example, at energies typically supplied by a subject's inhalation. Such energies are referred to herein as “low.” As used herein, “low energy administration” refers to administration wherein the energy applied to disperse and/or inhale the particles is in the range typically supplied by a subject during inhaling.

Preferred embodiments of the invention are described in the following example. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Example

### Stimulation of Amino Acids Availability and Metabolism in the Hypothalamus Regulates Glucose Production

Here we examined whether selective gluconeogenic and ketogenic amino acids contribute to the hypothalamic sensing of nutrients. We also investigated the mechanisms responsible for the amino acid-dependent signals.

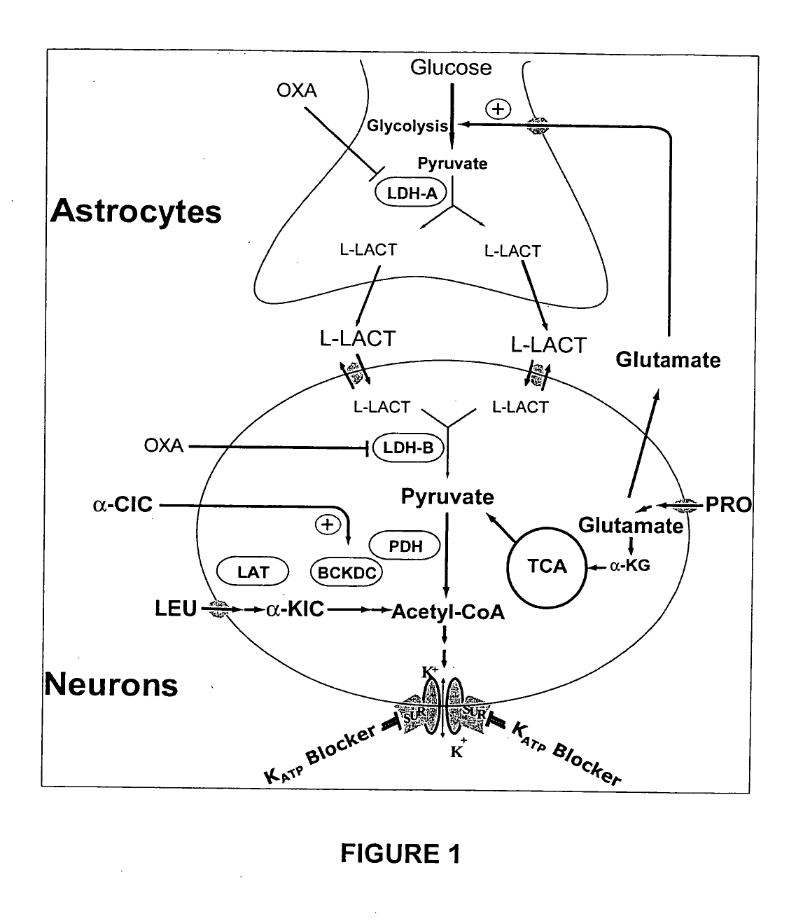

We hypothesized that the hypothalamic sensing of various classes of amino acids is a novel mechanism by which nutrients can regulate endogenous glucose production. In this regard, gluconeogenic amino acids such as proline can enter the tricarboxylic acids (TCA) cycle via conversion to TCA intermediates such as α-ketoglutarate and aspartate (FIG. 1). Additionally, particularly in neurons, proline and other gluconeogenic amino acids are also rapidly converted to glutamate, a main amino acid neurotransmitter. Glutamate has been in turn shown to activate glucose metabolism in astrocytes leading to increased availability of lactate. We had previously proposed that an increase in neuronal pyruvate metabolism is a hypothalamic signal of nutrient availability that can be generated by the increased formation of lactate (FIG. 1). Thus, the nutritional signal generated by the increased hypothalamic availability of gluconeogenic amino acids may require their conversion to glutamate and the stimulation of lactate formation in astrocytes. In this regard, the neuronal enzyme, lactic dehydrogenase B (LDH-B), regulates the entry of lactate in the tricarboxylic acids (TCA) cycle via its conversion to pyruvate. On the other hand ketogenic amino acids can directly generate acetyl-CoA that can in turn be oxidized to CO₂ or utilized within other metabolic pathways including the first step in the de novo lipid synthesis (i.e., the formation of malonyl-CoA). To begin unraveling the role of amino acid metabolism in the hypothalamic sensing of nutrients, we first asked whether a primary increase in the hypothalamic levels of either the gluconeogenic amino acids proline or glutamate, or the product of ketogenic amino acid metabolism α-keto-isocaproic acid (KIC) is sufficient to inhibit endogenous glucose production.

### Central Administration of Proline Inhibits Glucose Production.

To test this hypothesis we asked whether a primary increase in hypothalamic proline is per se sufficient to inhibit glucose production, and whether this effect requires metabolism of lactate to pyruvate. Experiments were designed to examine the effect of central administration of proline on whole body insulin action. In order to selectively increase the central availability of proline, we infused this amino acid via a chronic cannula implanted in the third cerebral ventricle (ICV) of conscious rats.

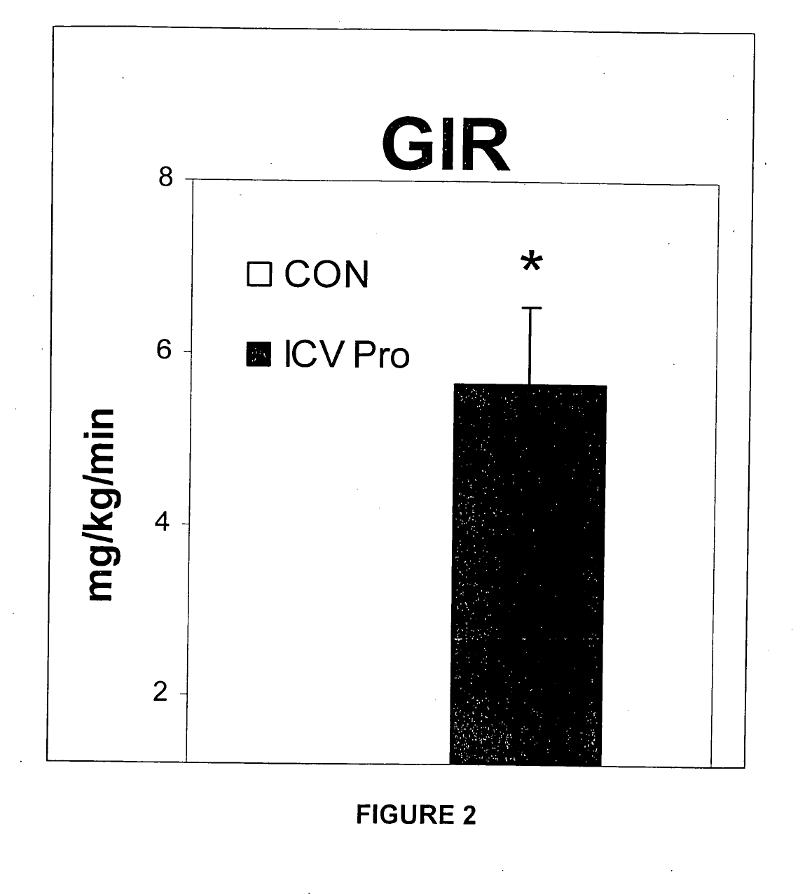

The central infusion of proline was sufficient to markedly diminish endogenous glucose production (FIG. 2). Proline (PRO, 3 mM solution at 5 μl/h) or vehicle (CON) was infused ICV for 6 hr in chronically catheterized Sprague Dawley rats. Insulin action was assessed by a combination of ICV infusions with systemic pancreatic-insulin clamp studies in order to control for the circulating levels of glucoregulatory hormones. As expected in the presence of ˜basal circulating insulin levels (clamp procedure), the rate of glucose infusion (GIR) required to maintain euglycemia was marginal in ICV control studies (0.6±0.4 mg/kg/min). By contrast, following ICV infusion of proline glucose had to be infused at the rate of 5.7±0.9 mg/kg/min in order to prevent hypoglycemia. Thus, increased central availability of proline in the presence of fixed and basal insulin concentrations stimulates insulin action on glucose homeostasis.

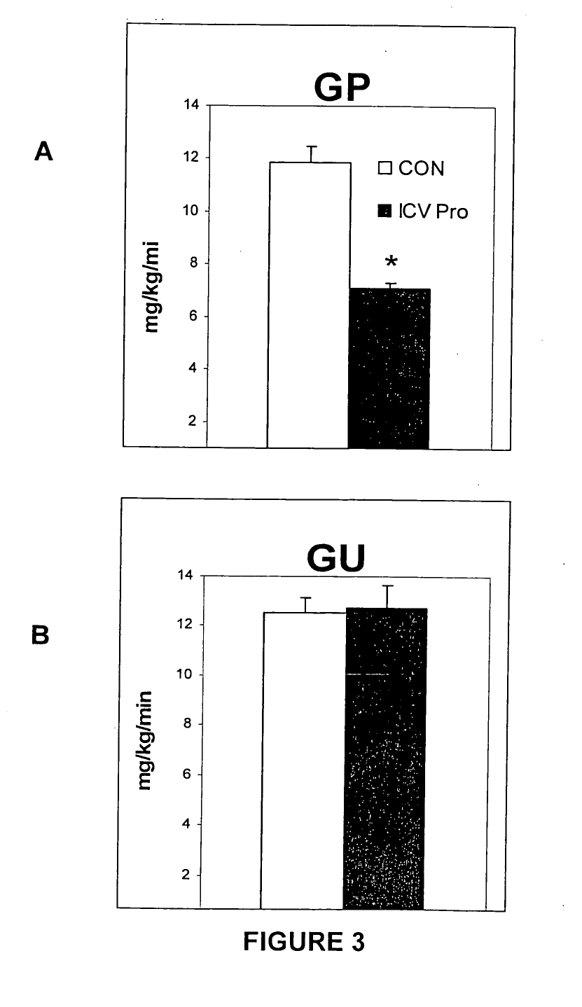

This stimulatory effect on glucose homeostasis could be due to either increased disposal of glucose (glucose uptake, GU) or to a diminished production of glucose (GP) by the liver (FIG. 3). The rate of glucose uptake was not significantly affected by ICV treatments. Conversely, ICV proline markedly and significantly decreased endogenous glucose production (by 36±2%).

This marked decrease in the rate of glucose production completely accounted for the effect of ICV proline on whole body glucose metabolism. On the basis of these results, we conclude that the central administration of proline is sufficient to markedly suppress endogenous glucose production.

### Metabolic Flux Through LDH is Required for the Central Effects of Proline on Glucose Production.

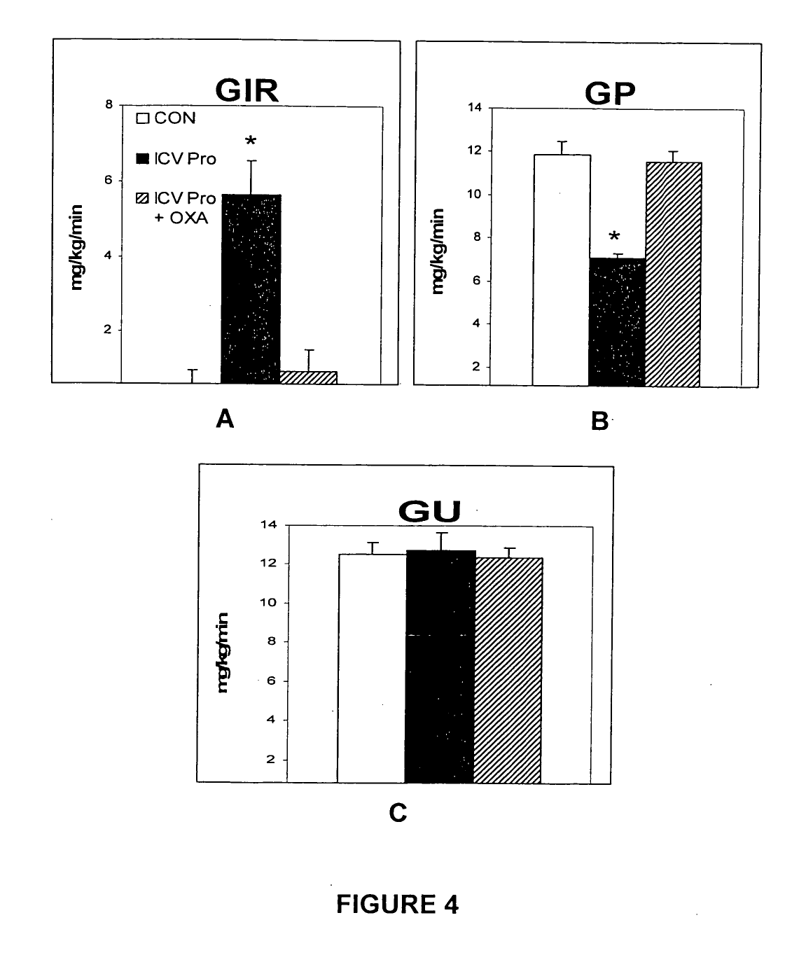

We next examined whether the effect of central proline on glucose production requires the inter-conversion between lactate and pyruvate catalyzed by LDH. In light of the ‘astrocyte-neuron lactate shuttle hypothesis’ (Pellerin and Magistretti, 2004), we proposed that central administration of proline leads to increased glutamate synthesis results in the increased formation of lactate (increased glycolysis) in astro-glia (Pellerin and Magistretti, 2004). Based on our previous work, the increased availability of lactate would in turn be expected to induce robust changes in liver glucose fluxes. Consistent with this hypothesis, impeding the conversion of glucose to lactate in astrocytes or of lactate to pyruvate in neurons within the hypothalamus should inhibit the potent metabolic effects of central proline on liver glucose fluxes. Oxamate (OXA) is a competitive inhibitor of LDH (Brooks et al., 1999). Thus, to test this hypothesis we next co-infused ICV oxamate (50 mM; 3 μl bolus; 5 μl/h) with proline in order to examine whether lactate metabolism is required for the effects of proline and presumably of other gluconeogenic amino acids on glucose production. During pancreatic clamps, ICV oxamate negated the effects of ICV proline on the rate of glucose infusion and on liver glucose fluxes (FIG. 4). Oxamate per se had no effects on these parameters (data not shown). These data indicate that hypothalamic metabolism of lactate to pyruvate is a required biochemical step for the potent effects of central proline on liver glucose metabolism.

Administration of Glutamate within the Mediobasal Hypothalamus is Per Se Sufficient to Inhibit Glucose Production.

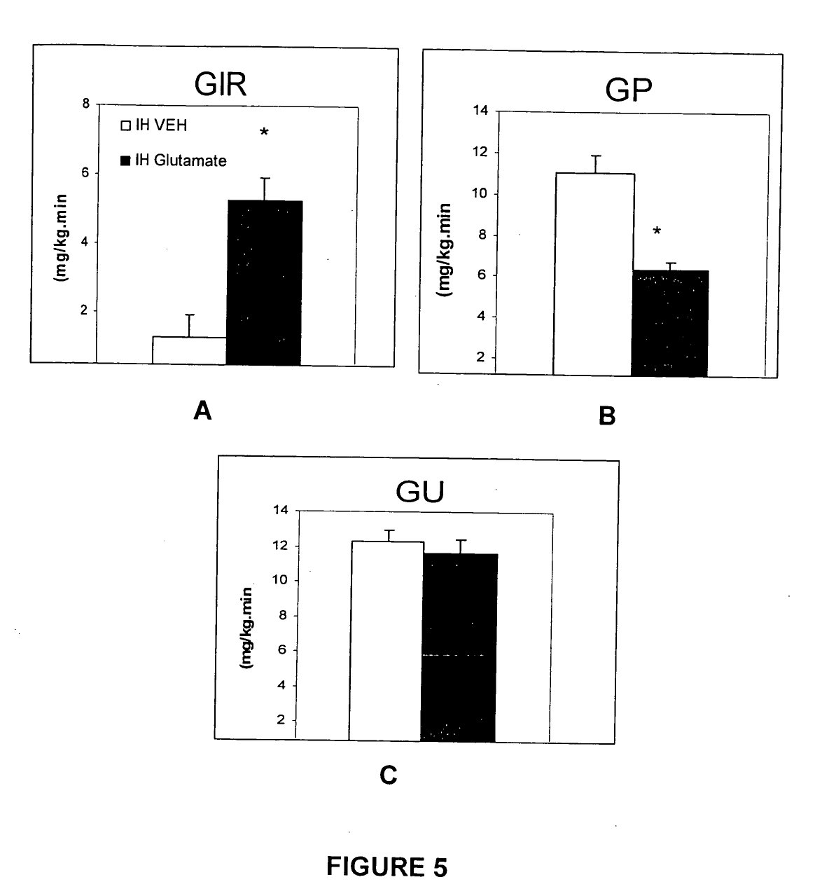

Since the flux through LDH was required for the effects of ICV proline on glucose production, we postulated that the nutritional signal generated by the increased hypothalamic availability of gluconeogenic amino acids may require their conversion to glutamate and the stimulation of lactate formation in astrocytes. Thus, we next tested whether the intra-hypothalamic (IH) administration of glutamate could recapitulate the effects of ICV proline on glucose production. Indeed, the IH administration of L-glutamate (1 mM, 0.33 μl/h) decreased circulating glucose (from 159±3 mg/dl to 136±2) (FIG. 5). In the presence of fixed and basal circulating insulin levels (pancreatic clamp), the rate of glucose infusion required to maintain plasma glucose to its basal levels was significantly increased in rats acutely infused with L-glutamate within the parenchyma of the mediobasal hypothalamus. A marked suppression of glucose production entirely accounted for the increase in the rate of glucose infusion. Thus, ICV proline administration regulates circulating glucose levels and liver glucose fluxes and these effects require its conversion to glutamate and the metabolism of lactate through LDH.

### Central Administration of α-Ketoisocaproic Acid (KIC) Inhibits Glucose Production.

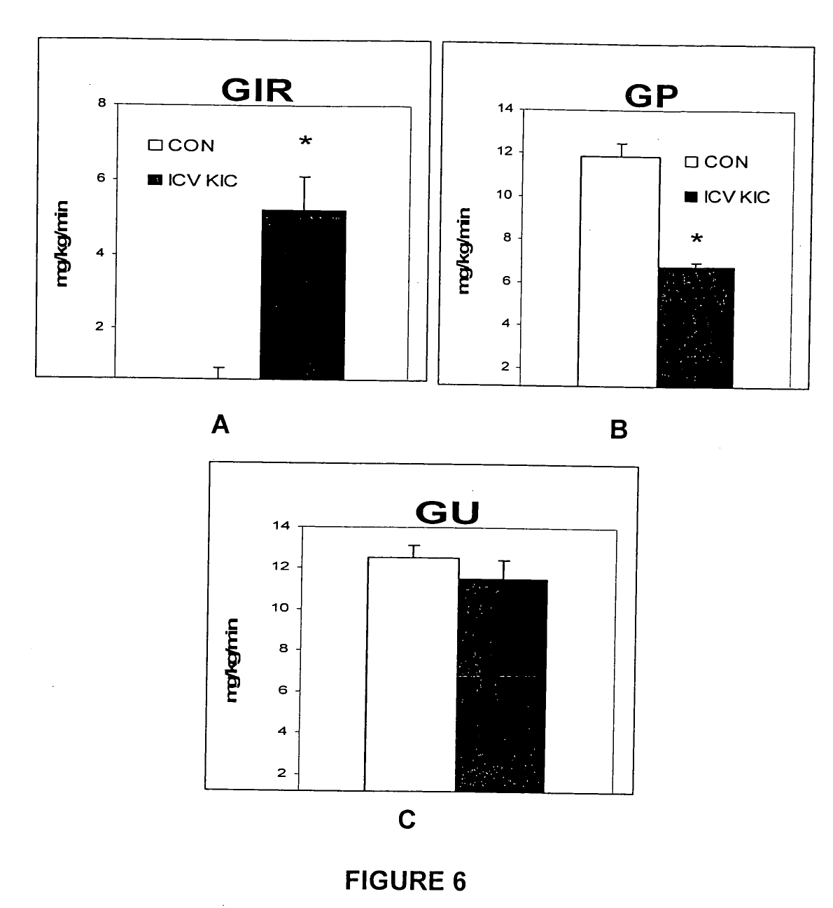

The entry of ketogenic amino acids into the oxidative pathway requires their conversion to the respective keto acid a reaction that is catalyzed by various amino acid aminotransferases. The keto acid product generated by leucine aminotransferase from leucine is α-ketoisocaproic acid (KIC) that can then be converted to acetyl-CoA by the consecutive action of five enzymes. The rate-limiting step in this process is the oxidative decarboxylation of KIC catalyzed by the branched-chain ketoacid dehydrogenase complex or BCKDC. This enzyme is inhibited by phosphorylation by a BCKDC kinase. Importantly, BCKDC can be robustly activated by α-chloroisocaproic acid (α-CIC) via inhibition of the BCKDC kinase (Harris et al., 1982). To test whether the central metabolism of ketogenic amino acids such as leucine could also lead to inhibition of glucose production, we next asked whether a primary increase in hypothalamic KIC is per se sufficient to inhibit glucose production, and whether this effect also requires metabolism of lactate to pyruvate and activation of ATP-dependent potassium channels (K_{ATP}). Experiments were designed to examine the effect of central administration of KIC on whole body insulin action. In order to selectively increase the central availability of KIC, we infused this amino acid via a chronic cannula implanted in the third cerebral ventricle (ICV) of conscious rats. The central infusion of KIC was sufficient to markedly diminish endogenous glucose production. KIC (3 mM solution at 5 μl/h) or vehicle (CON) was infused ICV for 6 hr in chronically catheterized Sprague Dawley rats. Insulin action was assessed by a combination of ICV infusions with systemic pancreatic-insulin clamp studies in order to control for the circulating levels of glucoregulatory hormones. As expected in the presence of ˜basal circulating insulin levels (clamp procedure), the rate of glucose infusion (GIR) required to maintain euglycemia was marginal in ICV control studies (FIG. 6). By contrast, following ICV infusion of KIC glucose had to be infused at the rate of 5.2±0.7 mg/kg/min in order to prevent hypoglycemia. Thus, increased central availability of KIC in the presence of fixed and basal insulin concentrations stimulates insulin action on glucose homeostasis.

This stimulatory effect on glucose homeostasis could be due to either increased disposal of glucose (glucose uptake) or to a diminished production of glucose by the liver. The rate of glucose uptake was not significantly affected by ICV treatments. Conversely, ICV KIC markedly and significantly decreased endogenous glucose production (by 40±4%). This marked decrease in the rate of glucose production completely accounted for the effect of ICV KIC on whole body glucose metabolism. On the basis of these results, we conclude that the central administration of KIC is sufficient to markedly suppress endogenous glucose production.

Administration of KIC within the Mediobasal Hypothalamus is Sufficient to Suppress Glucose Production.

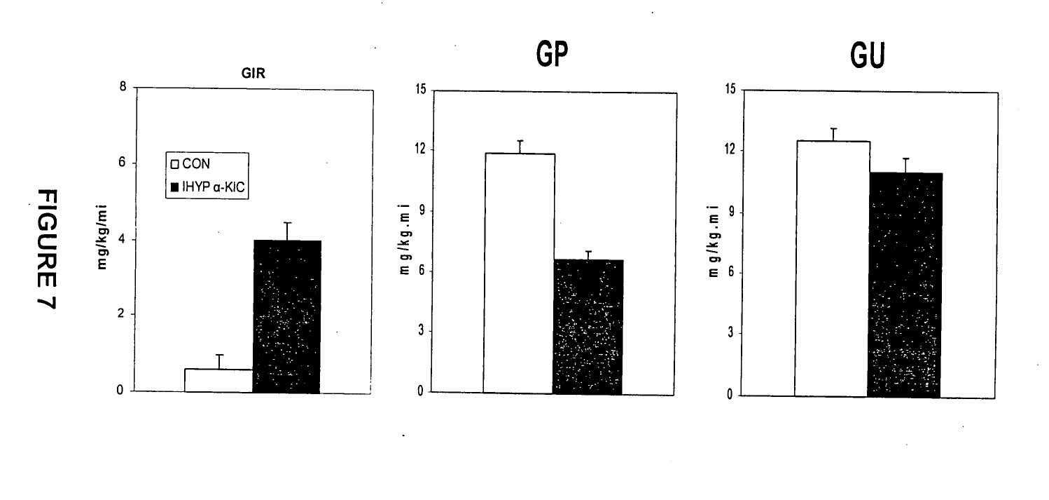

We next aimed to localize the central effects of KIC on liver glucose production. Toward this end, we administered 3 mM KIC (0.33 μl/h) bilaterally within the parenchyma of the medial hypothalamus. The total amount of KIC given during these experiments was 15 fold lower than that used in the ICV experiments. The correct placement of the hypothalamic cannulae was verified via the infusion of radiolabeled KIC as previously reported. During pancreatic clamps, the intrahypothalamic (LH) administration of KIC resulted in an increase in the rate glucose infusion required to maintain basal glucose levels (FIG. 7). The latter increase was due to suppression of liver glucose production while glucose utilization (GU) was not altered (FIG. 7). Thus, activation of a KIC-dependent pathway within the mediobasal hypothalamus is sufficient to inhibit glucose production. These data indicate that the mediobasal hypothalamus (arcuate nucleus) is the main site responsible for the effects of KIC on liver glucose homeostasis.

### Metabolic Flux Through LDH is not Required for the Central Effects of KIC on Glucose Production.

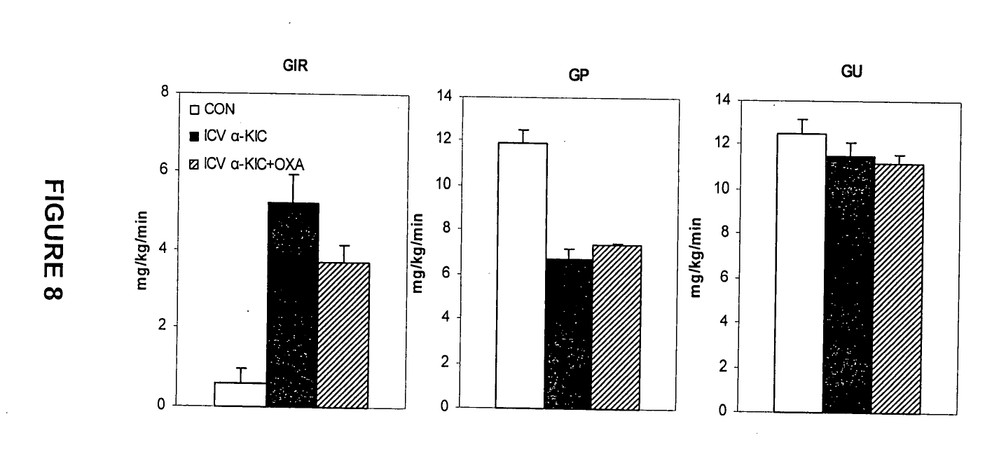

Since the central effects of gluconeogenic amino acids on glucose production required the inter-conversion between lactate and pyruvate catalyzed by LDH, we next examined whether the hypothalamic effects of KIC on glucose production also require flux through LDH. Oxamate is a competitive inhibitor of LDH (Harris et al., 1982). Thus, to test this hypothesis we next co-infused ICV oxamate (50 mM; 3 μl bolus; 5 μl/h) with KIC in order to examine whether lactate metabolism is also required for the effects of KIC and presumably of other ketogenic amino acids on glucose production. During pancreatic clamps, ICV oxamate failed to modify the effects of ICV KIC on the rate of glucose infusion and on liver glucose fluxes (FIG. 8). Oxamate per se had no effects on these parameters (data not shown). These data differ from those obtained with proline and indicate that the hypothalamic metabolism of lactate to pyruvate is not a required biochemical step for the potent effects of central KIC on liver glucose metabolism.

### Activation of K_{ATP} Channels is Required for the Central Effects of KIC on Glucose Production.

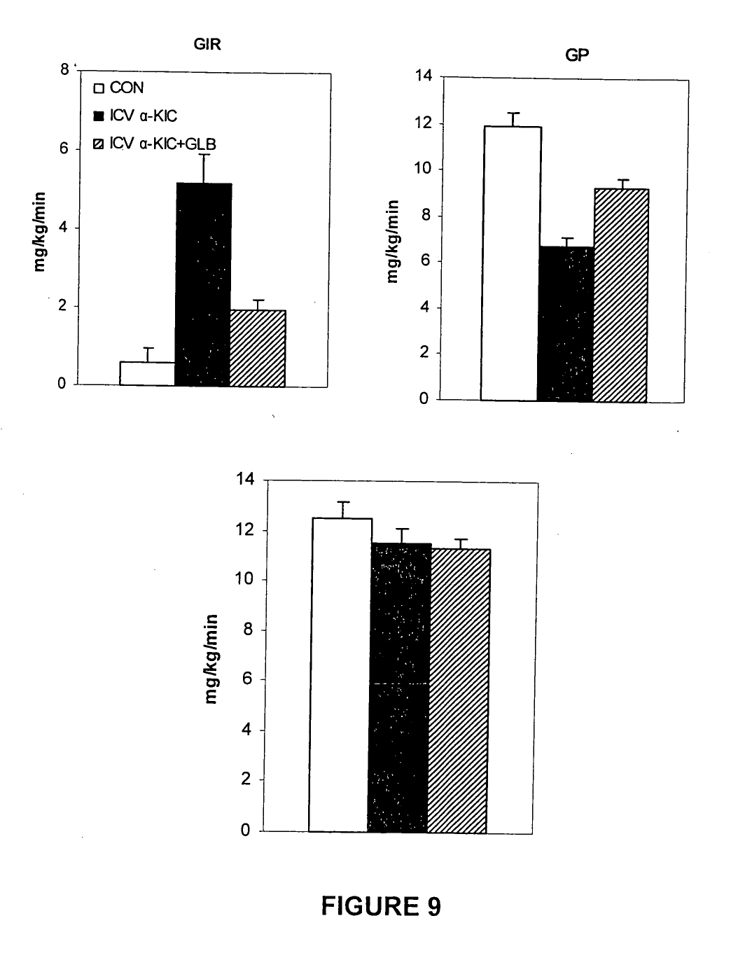

We have previously shown that the central antagonism of K_{ATP} channels activity abolished the suppressive effects of central and circulating insulin, lipids, and glucose on liver glucose fluxes (Obici et al., 2002; Lam et al., 2005). We hypothesized that this is a required step for the effects of KIC on liver glucose fluxes as well. To test this hypothesis, we co-infused ICV the K_{ATP} blocker glibenclamide (GLI, 100 μM; 5 μl/h) and KIC. Indeed, ICV infusion of the K_{ATP} blocker negated the suppressive effects of ICV KIC on the rate of glucose infusion and on liver glucose fluxes (FIG. 9). ICV infusion of K_{ATP}, blocker per se had no effects on these parameters. Taken together these findings support the postulate that hypothalamic sensing of ketogenic amino acids play a role in the regulation of liver glucose fluxes and that these central effects require activation of hypothalamic K_{ATP} channels but do not require stimulation of the astrocyte-neuron lactate shuttle.

Finally, direct activation of BCKDC by intrahypothalamic infusion of its activator α-chloroisocaproic acid (α-CIC) was per se sufficient to lower blood glucose levels and decrease endogenous glucose production in conscious rats.

Increased rates of endogenous glucose production lead to fasting hyperglycemia in diabetes mellitus. In summary, the hypothalamic sensing of gluconeogenic and ketogenic amino acids is likely to contribute to the central control of endogenous glucose production. Neuronal lactate metabolism plays a pivotal role in mediating the metabolic impacts of the hypothalamic sensing of gluconeogenic but not of ketogenic amino acids. Conversely, direct activation of hypothalamic KIC metabolism is sufficient to lower blood glucose via inhibition of glucose production. In conclusion, these results provide strong evidence in support of the notion that any strategy that increases the hypothalamic metabolism of either gluconeogenic or ketogenic amino acids will lower blood glucose concentrations via decreased rates of glucose production by the liver. Several alternative strategies to modulate hypothalamic metabolism of amino acids can be proposed since they represent viable biochemical approaches to the regulation of glucose metabolism via this novel central mechanism.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1. (canceled)

2. A method of reducing peripheral blood glucose levels or food intake or glucose production in a mammal, the method comprising administering a compound to the mammal in an amount effective to reduce blood glucose levels or food intake or glucose production in the mammal, wherein the compound is an amino acid, an amino acid analog, an immediate precursor to an amino acid, an amino acid-increasing molecule, a molecule that metabolizes an amino acid toward acetyl-CoA, or an inhibitor of an enzyme that decreases the activity of a molecule that metabolizes the amino acid toward acetyl-CoA.

3-7. (canceled)

8. A method of inhibiting gluconeogenesis in the liver of a mammal, the method comprising administering a compound to the mammal in an amount effective to inhibit gluconeogenesis in the liver of the mammal, wherein the compound is an amino acid, an amino acid analog, an immediate precursor to an amino acid, an amino acid-increasing molecule, a molecule that metabolizes an amino acid toward acetyl-CoA, or an inhibitor of an enzyme that decreases the activity of a molecule that metabolizes the amino acid toward acetyl-CoA.

9. (canceled)

10. A method of decreasing serum triglyceride levels or very low density lipoprotein (VLDL) levels in a mammal, the method comprising administering a compound to the mammal in an amount effective to decrease serum triglyceride levels or very low density lipoprotein (VLDL) levels in the mammal, wherein the compound is an amino acid, an immediate precursor to an amino acid, an amino acid analog, an amino acid-increasing molecule, a molecule that metabolizes an amino acid toward acetyl-CoA, or an inhibitor of an enzyme that decreases the activity of a molecule that metabolizes the amino acid toward acetyl-CoA.

11-12. (canceled)

13. The method of claim 2, wherein the mammal has at least one condition selected from the group consisting of obesity, type 2 diabetes, type 1 diabetes, insulin resistance, leptin resistance, metabolic syndrome, gonadotropin deficiency, amenorrhea, lactic acidosis, or polycystic ovary syndrome.

14-28. (canceled)

29. The method of claim 2, wherein the compound is the amino acid.

30. The method of claim 29, wherein the amino acid is a glucogenic amino acid.

31. The method of claim 30, wherein the glucogenic amino acid is alanine, arginine, asparagine, aspartate, cysteine, glycine, histidine, methionine, proline, serine, threonine, glutamine, glutamate, valine, isoleucine, phenylalanine, tryptophan, tyrosine, N-formaminoglutamate, homoserine, arginosuccinate, cystathionine, citrulline, homocysteine, ornithine, cysteinesulfinate, S-adenosylmethionine, S-adenyosylhomocysteine, glutamate-γ-semialdehyde, or 2-amino-3-ketobutyrate.

32. The method of claim 30, wherein the glucogenic amino acid is glutamate or proline.

33. The method of claim 29, wherein the amino acid is a ketogenic amino acid.

34. The method of claim 33, wherein the ketogenic amino acid is isoleucine, leucine, tryptophan, lysine, phenylalanine, tyrosine, aspartate β-semialdehyde, β-aspartylphosphate, saccharopine, α-aminoadipate δ-semialdehyde, or α-aminoadipate.

35. The method of claim 33, wherein the ketogenic amino acid is leucine.

36. The method of claim 2, wherein the compound is an immediate precursor of the amino acid.

37. The method of claim 36, wherein the immediate precursor of the amino acid is α-ketoglutarate, indole, α-ketobutarate, L-histadinol, phenylpyruvate, 4-hydroxyphenylpyruvate, methyltetrahydrofolate, α-keto-β-methylvalerate, or α-ketoisovalerate.

38-40. (canceled)

41. The method of claim 40, wherein the compound is the amino acid-increasing molecule.

42. The method of claim 40, wherein the amino acid-increasing molecule is a glutamate dehydrogenase, a valine aminotransferase, an arginosuccinate lyase, a tyrosine transaminase, an aromatic amino acid transaminase, a tryptophan synthase, or a histidinol dehydrogenase.

43. (canceled)

44. The method of claim 2, wherein the compound is the molecule that metabolizes the amino acid toward acetyl-CoA.

45. The method of claim 44, wherein the molecule that metabolizes the amino acid toward acetyl-CoA is a threonine dehydrogenase, a tyrosine aminotransferase, a serine dehydratase, an acyl-CoA dehydrogenase, a branched chain amino acid transaminase, or a branched chain α-ketoacid dehydrogenase.

46. The method of claim 44, wherein the molecule that metabolizes the amino acid toward acetyl-CoA is a branched chain α-ketoacid dehydrogenase.

47. The method of claim 2, wherein the compound inhibits an enzyme that decreases the activity of the molecule that metabolizes the amino acid toward acetyl-CoA.

48. The method of claim 47, wherein the compound is α-chloroisocaproic acid (α-CIC).

49-53. (canceled)

54. The method of claim 2, wherein the compound is formulated in a pharmaceutical composition that enhances the ability of the compound to cross the blood-brain barrier of the mammal.

55. The method of claim 54, wherein the compound is administered in a manner that permits the compound to cross the blood-brain barrier of the mammal.

56. The method of claim 2, wherein the compound is administered directly to the brain of the mammal.

57. The method of claim 2, wherein the mammal is a human.

58-59. (canceled)

60. A method of increasing glucose production or food intake in a mammal, the method comprising administering a compound to the mammal, wherein administering the compound to the mammal increases glucose production or food intake in the mammal and
(a) causes an decrease in an amino acid in the hypothalamus of the mammal provided the decrease is not due to metabolism of the amino acid toward acetyl-CoA, or
(b) causes an decrease in metabolism of the amino acid or an α-keto acid corresponding to the amino acid toward acetyl-CoA in the hypothalamus of the mammal.

61-78. (canceled)
